(19) United States
(12) Patent Application Publication
(10) Pub. No.: US 2015/0197760 A1
(43) Pub. Date: Jul. 16, 2015

(54) PROMOTERS FOR EXPRESSING A GENE IN A CELL

(71) Applicant: DSM IP ASSETS B.V., Heerlen (NL)

(72) Inventors: Alrik Pieter Los, Echt (NL); Wilbert Herman Marie Heijne, Echt (NL); Herman Jan Pel, Echt (NL); Robbertus Antonius Damveld, Echt (NL); Brenda Vonk, Echt (NL)

(21) Appl. No.: 14/408,089

(22) PCT Filed: Jun. 17, 2013
(86) PCT No.: PCT/EP2013/062490

§ 371 Date: Dec. 15, 2014

(30) Foreign Application Priority Data
Jun. 19, 2012 (EP) 12172605.3

(51) Int. Cl. C12N 15/80; C12N 9/34

(52) U.S. Cl. CPC C12N 15/80; C12N 9/2428

(57) ABSTRACT

The present invention relates to isolated Rasamsonia promoter DNA sequences, to DNA constructs, vectors, and host cells comprising these promoters in operative association with coding sequences. The present invention also relates to methods for expressing a gene and/or producing a biological compound using the new promoters isolated. The present invention also relates to methods for altering the transcription level and/or regulation of an endogenous gene using the new promoter of the invention.

### FIELD OF THE INVENTION

The present invention relates to DNA sequences, in particular isolated promoters, and to DNA constructs, vectors, and host cells comprising these promoters in operative association with coding sequences. The present invention also relates to methods for expressing a gene and/or producing a biological compound.

### BACKGROUND OF THE INVENTION

Production of a recombinant biological compound in a host cell is usually accomplished by constructing an expression cassette in which the DNA coding for the biological compound is operably linked to a promoter suitable for the host cell. The expression cassette may be introduced into the host cell, by plasmid- or vector-mediated transformation. Production of the biological compound may then be achieved by culturing the transformed host cell under inducing conditions necessary for the proper functioning of the promoter contained in the expression cassette.

For each host cell, expression of a coding sequence which has been introduced into the host by transformation and production of a recombinant biological compound encoded by this coding sequence requires the availability of functional promoters. Numerous promoters are already known to be functional in various host cells. There are examples of cross-species use of promoters in fungal host cells: the promoter of the Aspergillus nidulans (A. nidulans gpdA gene is known to be functional in Aspergillus niger (A. niger) (J Biotechnol. 1991 January; 17(1):19-33. Intracellular and extracellular production of proteins in Aspergillus under the control of expression signals of the highly expressed A. nidulans gpdA gene. Punt P J, Zegers N D, Busscher M, Pouwels P H, van den Hondel C A.) Another example is the A. niger beta-xylosidase xlnD promoter used in A. niger and A. nidulans Transcriptional regulation of the xylanolytic enzyme system of Aspergillus, van Peij, NNME, PhD-thesis Landbouwuniversiteit Wageningen, the Netherlands, ISBN 90-5808-154-0 and the expression of the Escherichia coli beta-glucuronidase gene in A. niger, A. nidulans and Cladosporium fulvum as described in Curr Genet. 1989 March; 15(3):177-80: Roberts I N, Oliver R P, Punt P J, van den Hondel C A. “Expression of the Escherichia coli beta-glucuronidase gene in industrial and phytopathogenic filamentous fungi”.

No Rasamsonia emersonii promoters are used for recombinant product formation sofar and only cross-species use of promoters are used.

There is still a need for promoters for controlling the expression of introduced genes, for controlling the level of expression of endogenous genes, for controlling the regulation of expression of endogenous genes or for mediating the inactivation of an endogenous gene, or for producing polypeptides, or for combination of the previous applications. These promoters, preferably improved promoters, may for example be stronger than the previous known ones. They may also be inducible by a specific convenient substrate or compound. Knowing several functional promoters is also an advantage when one envisages simultaneously over expressing various genes in a single host. To prevent squelching (titration of specific transcription factors), it is preferable to use multiple distinct promoters, e.g. one specific promoter for each gene to be expressed.

### BRIEF DESCRIPTION OF THE INVENTION

According to a first aspect the present invention provides a Rasamsonia promoter DNA sequence, preferably A Rasamsonia emersonii promoter DNA sequence. more preferably linked to a coding sequence which can be overexpressed. Preferably the Rasamsonia promoter DNA of the invention is linked to a coding sequence which can be overexpressed. Advantageously the Rasamsonia promoter of the invention corresponds to a strong promoter and/or an inducible promoter.

According to another aspect the present invention provides a promoter DNA sequence such as:
(a) a DNA sequence as presented in the following list: SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:15, SEQ ID NO:16, or SEQ ID NO:17,
(b) a DNA sequence capable of hybridizing with the complement of the DNA sequence of (a), or
(c) a DNA sequence being at least 50% homologous to a DNA sequence of (a).

To another aspect invention provides a DNA construct comprising a promoter DNA sequence of the invention and a coding sequence in operative association with said promoter DNA sequence such that the coding sequence can be expressed under the control of the promoter DNA sequence.

To a further aspect the invention provides a host cell, preferably a fungal host cell, comprising the DNA construct of the invention. This host cell is preferably a transformed host cell such as a transformed fungal host cell, and is advantageously produced with recombinant techniques. Preferably the host cell is a cell from the genus Acremonium, Agaricus, Aspergillus, Aureobasidium, Chrysosporium, Coprinus, Cryptococcus, Filobasidium, Fusarium, Geosmithia, Humicola, Magnaporthe, Mucor, Myceliophthora, Neocallimastix, Neurospora, Paecilomyces, Penicillium, Piromyces, Panerochaete, Pleurotus, Rasamsonia, Schizophyllum, Talaromyces, Thermoascus, Thermomyces, Thielavia, Tolypocladium, or Trichoderma, preferably from the genus Rasamsonia, Aspergillus, Penicillium, Chrysosporium or Trichoderma, preferably Rasamsonia emersonii.

To still another aspect the invention provides a method for expression of a coding sequence in a suitable host cell comprising:

(a) providing a DNA construct of the invention,

(b) transforming a suitable host cell with said DNA construct, and

(c) culturing the suitable host cell under culture conditions conducive to expression of the coding sequence.

Furthermore the invention provides a method for the production of a biological compound in a suitable host cell comprising:

(a) providing a DNA construct of the invention,

(b) transforming a suitable host cell with said DNA construct, and

(c) culturing the suitable host cell under culture conditions conducive to expression of the coding sequence, and optionally

(d) recovering the biological compound from the culture broth.

Advantageously the biological compound produced is a polypeptide or metabolite.

Preferably in the method of the invention the polypeptide produced is encoded by the coding sequence present in the DNA construct of the invention.

Advantageously in the method of the invention the coding sequence present in the DNA construct encodes an enzyme which is optionally involved in the production of the metabolite.

Furthermore the present invention provides a DNA sequence encoding a glucoamylase comprising:

(a) a DNA sequence as presented in SEQ ID NO:23,

(b) a DNA sequence capable of hybridizing with the complement of the DNA sequence of (a),

(c) a DNA sequence being at least 50%, preferably at least 60%, more preferably at least 70%, even more preferably at least 80%, still more preferably at least 90% and most preferably at least 95% homologous to a DNA sequence of (a), or

(d) a DNA sequence encoding a glucoamylase and being at least 50%, preferably at least 60%, more preferably at least 70%, even more preferably at least 80%, still more preferably at least 90% and most preferably at least 95% homologous to SEQ ID NO:24.

A further embodiment of the invention provides a glucoamylase having DNA sequence being at least 50%, preferably at least 60%, more preferably at least 70%, even more preferably at least 80%, still more preferably at least 90% and most preferably at least 95% homologous to SEQ ID NO:24.

### DESCRIPTION OF THE FIGURES

FIG. 1 shows a schematic diagram of plasmid pENTRY-P6bleTtrpC-Pxeba7flagTgla, which is the basis for a promoter test construct in R. emersonii. The promoter test construct comprises the ble expression cassette consisting of the A. nidulans gpdA promoter (P6), ble coding region (ble) and A. nidulans TrpC terminator (TtrpC), a promoter of interest (Px), the EBA7-FLAG reporter coding region (eba7flag) and the A. niger glucoamylase terminator.

FIG. 2 shows the expression of FLAG-tagged R. emersonii beta-glucanase CEB protein (EBA7-FLAG) driven by 5 different R. emersonii promoters expressed in supernatants of R. emersonii cultures as detected by Western blotting using a FLAG-specific antibody. Lane 1: CbhI promoter, 100 times diluted supernatant; lane 2: CbhI promoter, undiluted supernatant; lanes 3 and 4: AXE promoter; lane 5: empty strain; lane 6: empty lane; lane 7: A. nidulans gpdA promoter; lane 8: BG promoter; lanes 9 and 10: CbhII promoter; lane 11: EG promoter, undiluted supernatant; lane 12: EG promoter, 10 times diluted supernatant; lane 13: empty strain.

FIG. 3 shows a schematic diagram of plasmid Te pep.bbn, which is the basis for a promoter test construct in R. emersonii that is targeted to the RePepA locus. The vector comprises a 1500 bp 5′ flanking region 1.5 kb upstream of the RePepA ORF for targeting in the RePepA locus, a lox66 site, the non-functional 5′ part of the ble coding region driven by the A. nidulans gpdA promoter (5′ble), and a ccdB gene.

FIG. 4 shows a schematic diagram of plasmid pEBA1006 that was used in bipartite gene-targeting method in combination with the pEBA528, pEBA529, pEBA530, pEBA531, pEBA532 and pEBA533 vectors with the goal to replace the RePepA ORF and approximately 1500 nucleotides upstream of the start ATG codon by the promoter-reporter expression cassette in Rasamsonia emersonii. The vector comprises the 3′ part of the ble coding region, the A. nidulans trpC terminator, a lox71 site, a 2500 bp 3′ flanking region of the RePepA ORF, and the backbone of pUC19 (Invitrogen, Breda, The Netherlands).

FIG. 5 shows a schematic diagram of plasmid pEBA528 that was used in bipartite gene-targeting method in combination with the pEBA1006 vector with the goal to replace the RePepA ORF and approximately 1500 nucleotides upstream of the start ATG codon by the promoter-reporter expression cassette in Rasamsonia emersonii. The vector comprises a 1500 bp 5′ flanking region 1.5 kb upstream of the RePepA ORF for targeting in the RePepA locus, the promoter-reporter expression cassette consisting of R. emersonii promoter 1, FLAG-tagged R. emersonii glucosamylase (AG-FLAG) and the A. nidulans amdS terminator (TamdS), a lox66 site, the non-functional 5′ part of the ble coding region driven by the A. nidulans gpdA promoter (5′ ble). The E. coli DNA was removed by digestion with restriction enzyme NotI, prior to transformation of the R. emersonii strains.

FIG. 6 shows a schematic diagram of plasmid pEBA1001. Part of the vector fragment was used in bipartite gene-targeting method in combination with the pEBA1002 vector with the goal to delete the ReKu80 ORF in Rasamsonia emersonii. The vector comprises a 2500 bp 5′ upstream flanking region, a lox66 site, the 5′ part of the ble coding sequence driven by the A. nidulans gpdA promoter and the backbone of pUC19 (Invitrogen, Breda, The Netherlands). The E. coli DNA was removed by digestion with restriction enzyme NotI, prior to transformation of the R. emersonii strains.

FIG. 7 shows a schematic diagram of plasmid pEBA1002. Part of the vector fragment was used in bipartite gene-targeting method in combination with the pEBA1001 vector with the goal to delete the ReKu80 ORF in Rasamsonia emersonii. The vector comprises the 3′ part of the ble coding region, the A. nidulans trpC terminator, a lox71 site, a 2500 bp 3′ downstream flanking region of the ReKu80 ORF, and the backbone of pUC19 (Invitrogen, Breda, The Netherlands). The E. coli DNA was removed by digestion with restriction enzyme NotI, prior to transformation of the R. emersonii strains.

FIG. 8 shows the strategy used to delete the ReKu80 gene of R. emersonii. The vectors for deletion of ReKu80 comprise the overlapping non-functional ble selection marker fragments (split marker) flanked by loxP sites and 5′ and 3′ homologous regions of the ReKu80 gene for targeting (1). The constructs integrate through triple homologous recombination (X) at the genomic ReKu80 locus and at the overlapping homologous non-functional ble selection marker fragment (2) and replaces the genomic ReKu80 gene copy (3). Subsequently, the selection marker is removed by transient expression of cre recombinase leading to recombination between the lox66 and lox71 sites resulting in the deletion of the ble gene with a remainder double-mutant lox72 site left within the genome (4). Using this overall strategy, the ReKu80 ORF is removed from the genome.

FIG. 9 shows a schematic diagram of plasmid pEBA513 for transient expression of cre recombinase in fungi. pEBA513 is a pAMPF21 derived vector containing the AMA1 region and the CAT chloramphenicol resistance gene. Depicted are the cre recombinase gene (cre) expression cassette, containing the A. niger glaA promoter (Pgla), cre recombinase coding region, and niaD terminator. In addition, the hygromycin resistance cassette consisting of the A. nidulans gpdA promoter (PgpdA), hygB coding region and the P. chrysogenum penDE terminator (TpenDE) is indicated.

FIG. 10 shows the expression of FLAG-tagged R. emersonii glucoamylase (AG-FLAG) driven by 6 different R. emersonii promoters expressed in supernatants of R. emersonii cultures as detected by Western blotting using a FLAG-specific antibody. The different lanes show AG-FLAG expression in supernatants of transformants expressing the following promoter-reporter expression constructs: lane 1: pEBA540 (carrying A. nidulans gpdA promoter); lane 2, pEBA528 (carrying R. emersonii promoter 1); lane 3: pEBA529 (carrying R. emersonii promoter 2); lane 4: pEBA530 (carrying R. emersonii promoter 3); lane 5: pEBA531 (carrying R. emersonii promoter 4); lane 6: pEBA532 (carrying R. emersonii promoter 5); lane 7: pEBA533 (carrying R. emersonii promoter 6); and lane 8: empty strain.

### LIST OF SEQUENCES

SEQ ID NO: 1 R. emersonii cellobiohydrolased promoter

SEQ ID NO: 2 R. emersonii acetyl xylan esterase promoter

SEQ ID NO: 3 R. emersonii endoglucanase promoter

SEQ ID NO: 4 R. emersonii cellobiohydrolase-II promoter

SEQ ID NO: 5 R. emersonii beta-glucosidase promoter

SEQ ID NO: 6 A. nidulans gpdA promoter

SEQ ID NO: 7 R. emersonii RePepA (genomic sequence including flanks)

SEQ ID NO: 8 R. emersonii RePepA (cDNA)

SEQ ID NO: 9 R. emersonii RePepA (protein)

SEQ ID NO: 10 A. nidulans gpdA promoter and 5′ part of the ble coding region

SEQ ID NO: 11 3′ part of the ble coding region and A. nidulans TrpC terminator

SEQ ID NO: 12 R. emersonii promoter 1

SEQ ID NO: 13 R. emersonii promoter 2

SEQ ID NO: 14 R. emersonii promoter 3

SEQ ID NO: 15 R. emersonii promoter 4

SEQ ID NO: 16 R. emersonii promoter 5

SEQ ID NO: 17 R. emersonii promoter 6

SEQ ID NO: 18 FLAG-tagged R. emersonii glucoamylase (protein)

SEQ ID NO: 19 FLAG-tagged R. emersonii glucoamylase (DNA, coding region) and A. nidulans AmdS terminator

SEQ ID NO: 20 ReKu80 genomic sequence, coding region with flanks

SEQ ID NO: 21 ReKu80 cDNA sequence

SEQ ID NO: 22 ReKu80 protein sequence

SEQ ID NO: 23 ReGIa cDNA sequence

SEQ ID NO: 24 ReGIa protein sequence

### DETAILED DESCRIPTION OF THE INVENTION

Nowadays genomics projects use functional genomics approaches to identify new fungal enzymes for industrial and environmental applications. Genome DNA sequences are annotated on basis of publically known sequences. Many enzymes appear to be highly conserved during the evolution of the microorganism of origin. However for promoters hardly any conservation is noticed, identities of less than 5% are common even in case of closely related species, Therefore other strategies have to be developed to find new and effective promoters.

In the context of this invention, a promoter DNA sequence is a DNA sequence, which is capable of controlling the expression of a coding sequence, when this promoter DNA sequence is in operative association with this coding sequence. The term “in operative association” is defined herein as a configuration in which a promoter DNA sequence is appropriately placed at a position relative to a coding sequence such that the promoter DNA sequence directs the production of the product encoded by the coding sequence.

The term “coding sequence” is defined herein as a nucleic acid sequence that is transcribed into mRNA, which is translated into a polypeptide when placed under the control of the appropriate control sequences. The boundaries of the coding sequence are generally determined by the ATG start codon, which is normally the start of the open reading frame at the 5′ end of the mRNA and a transcription terminator sequence located just downstream of the open reading frame at the 3′ end of the mRNA. A coding sequence can include, but is not limited to, genomic DNA, cDNA, semisynthetic, synthetic, and recombinant nucleic acid sequences.

More specifically, the term “promoter” is defined herein as a DNA sequence that binds the RNA polymerase and directs the polymerase to the correct downstream transcriptional start site of a coding sequence encoding a polypeptide to initiate transcription. RNA polymerase effectively catalyzes the assembly of messenger RNA complementary to the appropriate DNA strand of the coding region. The term “promoter” will also be understood to include the 5′ non-coding region (between promoter and translation start) for translation after transcription into mRNA, cis-acting transcription control elements such as enhancers, and other nucleotide sequences capable of interacting with transcription factors.

The term “strong promoter” is defined herein as a promoter which gives more expression of a reporter protein compared to the A. nidulans gpdA promoter in a suitable nutrient medium containing 2.4% glucose or 2% cellulose as carbon source under suitable growth conditions. Examples of suitable reporter proteins are FLAG-tagged endoglucanase (described in Example 2) and FLAG-tagged glucoamylase (described in Example 5). Preferably, one copy of the promoter-reporter constructs is integrated into a specific locus to prevent differences in expression by copy number or position of integration into the genome (described in Example 5). Suitable nutrient media and growth conditions to compare promoter activities are dependent on the host. For example, the cells may be cultivated by shake flask cultivation, small-scale or large-scale fermentation (including continuous, batch, fed-batch, or solid state fermentations) in laboratory or industrial fermentors performed in a suitable medium and under conditions allowing the promoter-reporter gene to be expressed. The cultivation takes place in a suitable nutrient medium comprising carbon and nitrogen sources and inorganic salts, using procedures known in the art (for filamentous fungal hosts see, e.g., Bennett, J. W. and LaSure, L., eds., More Gene Manipulations in Fungi, Academic Press, CA, 1991). Examples of specific conditions to determine the activity of promoters in Rasamsonia are described in Example 2 and Example 5. Examples of suitable nutrient media and growth conditions in Trichoderma are described in Zou et al., 2012. Construction of a cellulase hyper-expression system in Trichoderma reesei by promoter and enzyme engineering. Microb Cell Fact., 2012 Feb. 8; 11(1): 21; and examples of suitable nutrient media and growth conditions for Aspergillus condition are described in EP 635 574.

The term “inducable promoter” is defined as a promoter which activity is induced by the presence or absence of biotic or abiotic factors, such as compounds derived from enzymatic hydrolysis of lignocellulose, metals, temperature or light. Examples of compounds derived from enzymatic hydrolysis of lignocellulose are sophorose, gentiobiose, cellobiose and xylose.

By overexpression of a coding sequence or gene of interest is meant an expression and/or secretion of a protein of interest which is novel or increased compared to the situation before, for example before the introduction of a promoter together with a coding sequence which enables expression in the parent cell,

A gene capable of high expression level, i.e. a highly expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1).

In a preferred embodiment the promoter is any Rasamsonia promoter. The selection of a specific promoter which is selected to transcribe a gene, is dependent on the medium conditions in which the promoter should be active. In addition, the strength of the promoter is a criterion of promoter selection. The strength of a promoter is dependent on the host strain and the fermentation conditions. Preferred promoters can be identified by growing the filamentous host under specific fermentation conditions and by quantifying transcript levels using for example microarray analysis, quantitative RT-PCR or RNA sequencing. Microarray analysis can be performed using standard methods known to the person skilled in the art for example by methods described in Kiryu et al., 2005. Extracting relations between promoter sequences and their strengths from microarray data. Bioinformatics 21 (7): 1062-1068. Sequencing of RNA can be performed using standard methods known to the person skilled in the art, for example using next generation sequencing technologies such as Illumina GA2, Roche 454, and the like, as reviewed in Pareek et al., 2011 Sequencing technologies and genome sequencing, J Appl Genetics 52:413-435. Alternatively, interesting promoters can be identified by proteomics studies, using MALDI-TOF analysis, LC-MS, or LC/MS-MS, in which promoters can be selected based on the amount of expressed protein.

By quantifying transcripts, it is possible to assess the promoter strength for a given condition. Comparing the strengths under different conditions allows the identification of condition-specific inducible promoters. Alternatively, promoters can be identified that active under different conditions and are constitutively active.

In a preferred embodiment, the promoter DNA sequence of the invention is a DNA sequence as presented in the following list: SEQ ID NO:1, SEQ ID NO:2, SEQ ID NO:3, SEQ ID NO:4, SEQ ID NO:5, SEQ ID NO:11, SEQ ID NO:13, SEQ ID NO:14, SEQ ID NO:15, SEQ ID NO:16, or SEQ ID NO:17.

According to another preferred embodiment, the promoter DNA sequence of the invention is a DNA sequence capable of hybridizing with a DNA sequence as presented in the following list: SEQ ID NO 1, SEQ ID NO:2, SEQ ID NO:3, SEQ ID NO:4, SEQ ID NO:5, SEQ ID NO:11, SEQ ID NO:13, SEQ ID NO:14, SEQ ID NO:15, SEQ ID NO:16, or SEQ ID NO:17, and which still retains promoter activity.

In the context of the invention, promoter activity is preferably determined by measuring the concentration of the protein(s) produced as a result of the expression of a coding sequence(s), which is (are) in operative association with the promoter. Alternatively the promoter activity is determined by measuring the enzymatic activity of the protein(s) encoded by the coding sequence(s), which is (are) in operative association with the promoter. According to a preferred embodiment, the promoter activity (and its strength) is determined by measuring the expression of the coding sequence of the lacZ reporter gene (In Luo (Gene 163 (1995) 127-131) or by measuring a FLAG-tagged protein such as FLAG-tagged glucoamylase (see Examples). According to another preferred embodiment, the promoter activity is determined by using the green fluorescent protein as coding sequence (In Microbiology. 1999 March; 145 (Pt 3):729-34. Santerre Henriksen A L, Even S, Muller C, Punt P J, van den Hondel C A, Nielsen J. Study). Additionally, promoter activity can be determined by measuring the mRNA levels of the transcript generated under control of the promoter. The mRNA levels can, for example, be measured through a Northern blot (J. Sambrook, E. F. Fritsch, and T. Maniatus, 1989, Molecular Cloning, A Laboratory Manual, 2d edition, Cold Spring Harbor, N.Y.). In all described assays to determine promoter activity, the activity of a promoter can compared to the activity of another promoter e.g. by placing identical reporter genes or coding sequences under control of the distinct promoters and measuring the promoter activities under identical conditions.

The present invention encompasses (isolated) promoter DNA sequences that hybridize under very low stringency conditions, preferably low stringency conditions, more preferably medium stringency conditions, more preferably medium-high stringency conditions, even more preferably high stringency conditions, and most preferably very high stringency conditions with the complementary strand of a nucleic acid probe that corresponds to:
a. nucleotides 1 to 1494 of SEQ ID NO:1 or SEQ ID NO:2, preferably nucleotides 100 to 1494, more preferably 200 to 1494, even more preferably 300 to 1494, even more preferably 350 to 1494 and most preferably 360 to 1494,
b. nucleotides 1 to 1482 of SEQ ID NO: 2, preferably nucleotides 100 to 1482, more preferably 200 to 1482, even more preferably 300 to 1482, even more preferably 350 to 1482 and most preferably 360 to 1482,
c. nucleotides 1 to 1503 of SEQ ID NO: 3 or SEQ ID NO:4 preferably 100 to 1503, more preferably 200 to 1503, even more preferably 300 to 1503, even more preferably 350 to 1503 and most preferably 360 to 1503,
d. nucleotides 1 to 1979 of SEQ ID NO:5, preferably nucleotides 100 to 1979, more preferably 200 to 1979, even more preferably 300 to 1979, even more preferably 350 to 1979 and most preferably 360 to 1979
e. nucleotides 1 to 1501 of SEQ ID NO:12, SEQ ID NO:13, SEQ ID NO:14, SEQ ID NO:16 or SEQ ID NO:17, preferably nucleotides 100 to 1501, more preferably 200 to 1501, even more preferably 300 to 1501, even more preferably 350 to 1501 and most preferably 360 to 1501, or
f. nucleotides 1 to 651 of SEQ ID NO:15, preferably 50 to 651, more preferably 100 to 651, even more preferably 150 to 651, even more preferably 200 to 651 and most preferably 250 to 651.
   The term complementary strand is known to the person skilled in the art and is described in J. Sambrook, E. F. Fritsch, and T. Maniatis, 1989, Molecular Cloning, A Laboratory Manual, 2d edition, Cold Spring Harbor, N.Y.

As used herein, the term “hybridizing” is intended to describe conditions for hybridization and washing under which nucleotide sequences at least about 60%, at least about 70%, at least about 80%, more preferably at least about 85%, even more preferably at least about 90%, more preferably at least 95%, more preferably at least 98% or more preferably at least 99% homologous to each other typically remain hybridized to each other.

A preferred, non-limiting example of such hybridization conditions are hybridization in 6× sodium chloride/sodium citrate (SSC) at about 45° C., followed by one or more washes in 1×SSC, 0.1% SDS at 50° C., preferably at 55° C., preferably at 60° C. and even more preferably at 65° C.

Highly stringent conditions include, for example, hybridizing at 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) residues, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

The subsequence of SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:15, SEQ ID NO:16 or SEQ ID NO:17, may be at least 100 nucleotides, preferably at least 200 nucleotides, more preferably at least 300 nucleotides, even more preferably at least 400 nucleotides and most preferably at least 500 nucleotides.

The nucleic acid sequence of SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:15, SEQ ID NO:16 or SEQ ID NO:17 or a subsequence thereof may be used to design a nucleic acid probe to identify and clone DNA promoters from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 15, preferably at least 25, and more preferably at least 35 nucleotides in length. Additionally, such probes can be used to amplify DNA promoters though PCR. Longer probes can also be used. DNA, RNA and Peptide Nucleid Acid (PNA) probes can be used. The probes are typically labelled for detecting the corresponding gene (for example, with @32 P, @33 P @3 H, @35 S, biotin, or avidin or a fluorescent marker). Such probes are encompassed by the present invention.

Thus, a genomic DNA or cDNA library prepared from such other organisms may be screened for DNA, which hybridizes with the probes described above and which encodes a polypeptide. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:15, SEQ ID NO:16 or SEQ ID NO:17, or a subsequence thereof, the carrier material may be used in a Southern blot.

For purposes of the present invention, hybridization indicates that the nucleic acid sequence hybridizes to a labeled nucleic acid probe corresponding to the complementory strand of nucleic acid sequence shown in SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:15, SEQ ID NO:16 or SEQ ID NO:17 under very low to very high stringency conditions. Molecules to which the nucleic acid probe hybridizes under these conditions are detected using for example an X-ray film. Other hybridisation techniques also can be used, such as techniques using fluorescence for detection and glass sides and/or DNA microarrays as support. An example of DNA microarray hybridisation detection is given in FEMS Yeast Res. 2003 December; 4(3):259-69 (Daran-Lapujade P, Daran J M, Kotter P, Petit T, Piper M D, Pronk J T. “Comparative genotyping of the Saccharomyces cerevisiae laboratory strains S288C and CEN.PK113-7D using oligonucleotide microarrays”. Additionally, the use of PNA microarrays for hybridization is described in Nucleic Acids Res. 2003 Oct. 1; 31(19):e119 (Brandt O, Feldner J, Stephan A, Schroder M, Schnolzer M, Arlinghaus H F, Hoheisel J D, Jacob A. PNA microarrays for hybridisation of unlabelled DNA samples.)

In a preferred embodiment, the nucleic acid probe is the nucleic acid sequence of SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:15, SEQ ID NO:16 or SEQ ID NO:17. In another preferred embodiment, the nucleic acid probe is the sequence having:
a. nucleotides 20 to 1480 of SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:16 or SEQ ID NO:17, more preferably nucleotides 500 to 1480 of SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:16 or SEQ ID NO:17, even more preferably nucleotides 800 to 1480 of SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:16 or SEQ ID NO:17, and most preferably nucleotides 900 to 1480 of SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:16 or SEQ ID NO:17, or
b. nucleotides 20 to 651 of SEQ ID NO:15, more preferably nucleotides 100 to 651 of SEQ ID NO: 15, even more preferably nucleotides 200 to 651 of SEQ ID NO: 15, and most preferably nucleotides 300 to 651 of SEQ ID NO: 15, or

Another preferred probe is the part of the DNA sequence immediately before the transcription initiation site.

For long probes of at least 100 nucleotides in length, very low to very high stringency conditions are defined as prehybridization and hybridization at 42 degrees Celsius in 5 times SSPE, 0.3% SDS, 200 microgram/ml sheared and denatured salmon sperm DNA, and either 25% formamide for very low and low stringencies, 35% formamide for medium and medium-high stringencies, or 50% formamide for high and very high stringencies, following standard Southern blotting procedures.

For long probes of at least 100 nucleotides in length, the carrier material is finally washed three times each for 15 minutes using 2 times SSC, 0.2% SDS preferably at least at 45 DEG C. (very low stringency), more preferably at least at 50 degrees Celsius (low stringency), more preferably at least at 55 degrees Celsius (medium stringency), more preferably at least at 60 degrees Celsius (medium-high stringency), even more preferably at least at 65 degrees Celsius (high stringency), and most preferably at least at 70 degrees Celsius (very high stringency).

For short probes which are about 15 nucleotides to about 70 nucleotides in length, stringency conditions are defined as prehybridization, hybridization, and washing post-hybridization at 5 degrees Celsius to 10 degrees Celsius below the calculated Tm using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1.times.Denhardt's solution, 1 mM sodium pyrophosphate, 1 mM sodium monobasic phosphate, 0.1 mM ATP, and 0.2 mg of yeast RNA per ml following standard Southern blotting procedures.

For short probes, which are about 15 nucleotides to about 70 nucleotides in length, the carrier material is washed once in 6 times SCC plus 0.1% SDS for 15 minutes and twice each for 15 minutes using 6 times SSC at 5 degrees Celsius to 10 degrees Celsius below the calculated Tm.

According to another preferred embodiment, SEQ ID NO 1, SEQ ID NO:2, SEQ ID NO:3, SEQ ID NO:4 SEQ ID NO:5, SEQ ID NO:12, SEQ ID NO:13, SEQ ID NO:14, SEQ ID NO:15, SEQ ID NO:16, or SEQ ID NO:17, is first used to clone the native gene, coding sequence or part of it, which is operatively associated with it. This can be done starting with SEQ ID NO 1, SEQ ID NO:2, SEQ ID NO:3, SEQ ID NO:4 SEQ ID NO:5, SEQ ID NO:12, SEQ ID NO:13, SEQ ID NO:14, SEQ ID NO:15, SEQ ID NO:16, or SEQ ID NO:17, or a subsequence thereof as earlier defined and using this sequence as a probe. The probe is hybridised to a cDNA or a genomic library of a given host, either Rasamsonia emersonii or any other host as defined in this application. Once the native gene or part of it has been cloned, it can be subsequently used itself as a probe to clone homologous genes thereof derived from other fungi by hybridisation experiments as described herein.

In the context of the invention, a homologous gene means a gene, which is at least 50% homologous (identical) to the native gene. Preferably, the homologous gene is at least 55% homologous, more preferably at least 60%, more preferably at least 65%, more preferably at least 70%, even more preferably at least 75% preferably about 80%, more preferably about 90%, even more preferably about 95%, even more preferably about 97%, even more preferably about 98%, even more preferably about 99%, and most preferably about 99.5% homologous to the native gene.

The sequence upstream of the coding sequence of the homologous gene is a promoter encompassed by the present invention. Alternatively, the sequence of the native gene, coding sequence or part of it, which is operatively associated with a promoter of the invention can be identified by using SEQ ID NO 1, SEQ ID NO:2, SEQ ID NO:3, SEQ ID NO:4 SEQ ID NO:5, SEQ ID NO:12, SEQ ID NO:13, SEQ ID NO:14, SEQ ID NO:15, SEQ ID NO:16, or SEQ ID NO:17, or a subsequence thereof as earlier defined to search genomic databases using for example an alignment or BLAST algorithm as described herein. This identified sequence subsequently can be used to identify orthologues or homologous genes in any other host as defined in this application. The sequence upstream the coding sequence of the identified orthologue or homologous gene is a promoter encompassed by the present invention.

According to another preferred embodiment, the promoter DNA sequence of the invention is a(n) (isolated) DNA sequence, which is at least 50% homologous (identical) to SEQ ID NO 1, SEQ ID NO:2, SEQ ID NO:3, SEQ ID NO:4 SEQ ID NO:5, SEQ ID NO:12, SEQ ID NO:13, SEQ ID NO:14, SEQ ID NO:15, SEQ ID NO:16, or SEQ ID NO:17. Preferably, the DNA sequence is at least 55% homologous, more preferably at least 60%, more preferably at least 65%, more preferably at least 70%, even more preferably at least 75% preferably about 80%, more preferably about 90%, even more preferably about 95%, even more preferably about 97%, even more preferably about 98%, even more preferably about 99%, and most preferably about 99.5% homologous to SEQ ID NO 1, SEQ ID NO:2, SEQ ID NO:3, SEQ ID NO:4 SEQ ID NO:5, SEQ ID NO:12, SEQ ID NO:13, SEQ ID NO:14, SEQ ID NO:15, SEQ ID NO:16, or SEQ ID NO:17.

For purposes of the present invention, the degree of homology (identity) between two nucleic acid sequences is preferably determined by the BLAST program. Software for performing BLAST analyses is publicly available through the National Center for Biotechnology Information (http://www.ncbi.nlm.nih.gov/). The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89: 10915 (1989)) alignments (B) of 50, expectation (E) of 10, M=5, N=−4, and a comparison of both strands.

The terms “homology”, “identity” or “percent identity” are used interchangeably herein. For the purpose of this invention, it is defined here that in order to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Preferably, the two sequences are the same length.

In another preferred embodiment, the promoter is a subsequence of SEQ ID NO 1, SEQ ID NO:2, SEQ ID NO:3, SEQ ID NO:4 SEQ ID NO:5, SEQ ID NO:12, SEQ ID NO:13, SEQ ID NO:14, SEQ ID NO:15, SEQ ID NO:16, or SEQ ID NO:17, the subsequence still having promoter activity. The subsequence preferably contains at least about 100 nucleotides, more preferably at least about 200 nucleotides, and most preferably at least about 300 nucleotides.

In another preferred embodiment, a subsequence is a nucleic acid sequence encompassed by SEQ ID NO 1, SEQ ID NO:2, SEQ ID NO:3, SEQ ID NO:4 SEQ ID NO:5, SEQ ID NO:12, SEQ ID NO:13, SEQ ID NO:14, SEQ ID NO:15, SEQ ID NO:16, or SEQ ID NO:17 except that one or more nucleotides from the 5′ and/or 3′ end have been deleted, said DNA sequence still having promoter activity.

In another preferred embodiment, the promoter subsequence is a ‘trimmed’ subsequence, i.e. a sequence fragment, which is upstream from translation start and/or from transcription start. An example of trimming a promoter and functionally analysing it is described in Gene. 1994 Aug. 5; 145(2):179-87: the effect of multiple copies of the upstream region on expression of the Aspergillus niger glucoamylase-encoding gene. Verdoes J C, Punt P J, Stouthamer A H, van den Hondel C A).

In another embodiment of the invention, the promoter DNA sequence is a variant of SEQ ID NO 1, SEQ ID NO:2, SEQ ID NO:3, SEQ ID NO:4 SEQ ID NO:5, SEQ ID NO:11, SEQ ID NO:13, SEQ ID NO:14, SEQ ID NO:15, SEQ ID NO:16, or SEQ ID NO:17.

The term “variant” or “variant promoter” is defined herein as a promoter having a nucleotide sequence comprising a substitution, deletion, and/or insertion of one or more nucleotides of a parent promoter, wherein the variant promoter has more or less promoter activity than the corresponding parent promoter. Such substitutions, deletions and/or insertions may very in length, for example from 1-1000 nucleotides, preferably 1-100 nucleotides, more preferably from 1-20 nucleotides, even more preferably from 1-10 nucleotides, still more preferably from 1-6 nucleotides, and most preferably from 1-3 nucleotides, still leading to a biologically active polynucleotide with promoter activity.

The term “variant promoter” will encompass natural variants and in vitro generated variants obtained using methods well known in the art such as classical mutagenesis, site-directed mutagenesis, and DNA shuffling. A variant promoter may have one or more mutations. Each mutation is an independent substitution, deletion, and/or insertion of a nucleotide.

According to a preferred embodiment, the variant promoter is a promoter, which has at least a modified regulatory site as compared to the promoter sequence first identified (SEQ ID NO 1, SEQ ID NO:2, SEQ ID NO:3, SEQ ID NO:4 SEQ ID NO:5, SEQ ID NO:12, SEQ ID NO:13, SEQ ID NO:14, SEQ ID NO:15, SEQ ID NO:16, or SEQ ID NO:17). Such a regulatory site can be removed in its entirety or specifically mutated as explained above. The regulation of such promoter variant is thus modified so that for example it is no longer induced by glucose. Examples of such promoter variants and techniques on how to obtain them are described in EP 673 429 or in WO 94/04673.

The promoter variant can be an allelic variant. An allelic variant denotes any of two or more alternative forms of a gene occupying the same chromosomal locus. Allelic variation arises naturally through mutation, and may result in polymorphism within populations. The variant promoter may be obtained by (a) hybridizing a DNA under very low, low, medium, medium-high, high, or very high stringency conditions with (i) SEQ ID NO 1, SEQ ID NO:2, SEQ ID NO:3, SEQ ID NO:4 SEQ ID NO:5, SEQ ID NO:12, SEQ ID NO:13, SEQ ID NO:14, SEQ ID NO:15, SEQ ID NO:16, or SEQ ID NO:17, (ii) a subsequence of (i) or (iii) a complementary strand of (i), (ii), and (b) isolating the variant promoter from the DNA. Stringency and wash conditions are as defined herein.

The promoter of the invention can be a promoter, whose sequence may be provided with linkers for the purpose of introducing specific restriction sites facilitating ligation of the promoter sequence with the coding region of the nucleic acid sequence encoding a polypeptide.

The sequence information as provided herein should not be so narrowly construed as to require inclusion of erroneously identified bases. The specific sequences disclosed herein can readily be used to isolate the original DNA sequence, preferably from a filamentous fungus, in particular Rasamsonia, and be subjected to further sequence analyses thereby identifying sequencing errors.

Unless otherwise indicated, all nucleotide sequences determined by sequencing a DNA molecule herein were determined using an automated DNA sequencer. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art.

The person skilled in the art is capable of identifying such erroneously identified bases and knows how to correct for such errors.

The present invention encompasses functional promoter equivalents typically containing mutations that do not alter the biological function of the promoter it concerns. The term “functional equivalents” also encompasses orthologues of the Rasamsonia DNA sequences. Orthologues of the Rasamsonia DNA sequences are DNA sequences that can be isolated from other organisms, other fungal species or strains and possess a similar or identical biological activity.

The promoter sequences of the present invention may be obtained from microorganisms of any genus. For purposes of the present invention, the term “obtained from” as used herein in connection with a given source shall mean that the polypeptide is produced by the source or by a cell in which a gene from the source has been inserted.

The promoter sequences may be obtained from a fungal source, preferably from a Rasamsonia strain, more preferably Rasamsonia emersonii.

Rasamsonia is a new genus comprising thermotolerant and thermophilic Talaromyces and Geosmithia species (J. Houbraken et al vida supra). Based on phenotypic, physiological and molecular data, Houbraken et al proposed to transfer the species T. emersonii, T. byssochlamydoides, T. eburneus, G. argillacea and G. cylindrospora to Rasamsonia gen. nov. Talaromyces emersonii, Penicillium geosmithia emersonii and Rasamsonia emersonii are used interchangeably herein.

It will be understood that for the aforementioned species, the invention encompasses the perfect and imperfect states, and other taxonomic equivalents, e.g., anamorphs, regardless of the species name by which they are known. Those skilled in the art will readily recognize the identity of appropriate equivalents. Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

Furthermore, promoter sequences according to the invention may be identified and obtained from other sources including microorganisms isolated from nature (e.g, soil, composts, water, etc.) using the above-mentioned probes. Techniques for isolating microorganisms from natural habitats are well known in the art. The nucleic acid sequence may then be derived by similarly screening a genomic DNA library of another microorganism. Once a nucleic acid sequence encoding a promoter has been detected with the probe(s), the sequence may be isolated or cloned by utilizing techniques which are known to those of ordinary skill in the art (see, e.g., Sambrook et al., 1989, supra).

In the present invention, the promoter DNA sequence may also be a hybrid promoter comprising a portion of one or more promoters of the present invention; a portion of a promoter of the present invention and a portion of another known promoter, e.g., a leader sequence of one promoter and the transcription start site from the other promoter; or a portion of one or more promoters of the present invention and a portion of one or more other promoters. The other promoter may be any promoter sequence, which shows transcriptional activity in the host cell of choice including a variant, truncated, and hybrid promoter, and may be obtained from genes encoding extracellular or intracellular polypeptides either homologous or heterologous to the host cell. The other promoter sequence may be native or foreign to the nucleic acid sequence encoding the polypeptide and native or foreign to the cell.

As a preferred embodiment, important regulatory subsequences of the promoter identified can be fused to other ‘basic’ promoters to enhance their promoter activity (as for example described in Mol Microbiol. 1994 May; 12(3):479-90. Regulation of the xylanase-encoding xlnA gene of Aspergillus tubigensis. de Graaff L H, van den Broeck H C, van Ooijen A J, Visser J.).

Other examples of other promoters useful in the construction of hybrid promoters with the promoters of the present invention include the promoters obtained from the genes for A. oryzae TAKA amylase, Rhizomucor miehei aspartic proteinase, A. niger neutral alpha-amylase, A. niger acid stable alpha-amylase, A. niger or Aspergillus awamori glucoamylase (glaA), A. niger gpdA, A. niger glucose oxidase goxC, Rhizomucor miehei lipase, A. oryzae alkaline protease, A. oryzae triose phosphate isomerase, A. nidulans acetamidase, and Fusarium oxysporum trypsin-like protease (WO 96/00787), as well as the NA2-tpi promoter (a hybrid of the promoters from the genes for A. niger neutral alpha-amylase and A. oryzae triose phosphate isomerase), Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase, and mutant, truncated, and hybrid promoters thereof. Other useful promoters for yeast host cells are described by Romanos et al., 1992, Yeast 8: 423-488.

In the present invention, the promoter DNA sequence may also be a “tandem promoter”. A “tandem promoter” is defined herein as two or more promoter sequences each of which is in operative association with a coding sequence and mediates the transcription of the coding sequence into mRNA.

The tandem promoter comprises two or more promoters of the present invention or alternatively one or more promoters of the present invention and one or more other known promoters, such as those exemplified above useful for the construction of hybrid promoters. The two or more promoter sequences of the tandem promoter may simultaneously promote the transcription of the nucleic acid sequence. Alternatively, one or more of the promoter sequences of the tandem promoter may promote the transcription of the nucleic acid sequence at different stages of growth of the cell or morphological different parts of the mycelia.

In the present invention, the promoter may be foreign to the coding sequence encoding a biological compound and/or the promoter may be foreign to the host cell. A variant, hybrid, or tandem promoter of the present invention will be understood to be foreign to a coding sequence encoding even if the wild-type promoter is native to the coding sequence or to the host cell.

A variant, hybrid, or tandem promoter of the present invention has at least about 20%, preferably at least about 40%, more preferably at least about 60%, more preferably at least about 80%, more preferably at least about 90%, more preferably at least about 100%, even more preferably at least about 200%, most preferably at least about 300%, and even most preferably at least about 400% of the promoter activity of the promoter having SEQ ID NO 1, SEQ ID NO:2, SEQ ID NO:3, SEQ ID NO:4 SEQ ID NO:5, SEQ ID NO:12, SEQ ID NO:13, SEQ ID NO:14, SEQ ID NO:15, SEQ ID NO:16 or SEQ ID NO:17. Promoter activity is preferably determined as described earlier in the description.

The invention further relates to a DNA construct comprising a (“a” is herein defined as “at least one”) promoter DNA sequence as defined above and a coding sequence in operative association with said promoter DNA sequence such that the coding sequence can be expressed under the control of the promoter DNA sequence. This may be tested in any suitable host cell. Alternatively, this may be tested in a suitable in vitro expression and/or translation system. The coding sequence may be obtained from any prokaryotic, eukaryotic, or other source. Alternatively, the coding sequence may be a synthetic, or partly synthetic sequence. The codon usage of the synthetic gene may have been optimized to match the codon usage of the host cell species to improve expression and/or secretion of the encoded biological substance. An example of codon usage optimization is described in WO 97/11086, where codon usage of plant polypeptides is optimized of expression in filamentous fungal cells. Preferably, the coding sequence encodes a biological compound. Two or more of these DNA constructs may be linked to form a new (tandem) DNA construct. This new (tandem) construct may comprise two or more of the DNA constructs which will for example comprise (promoter-open reading frame-terminator) linked to (promoter-open reading frame-terminator) which optionally may be linked to the next (promoter-open reading frame-terminator) unit. In case of for example 5 lined units, the DNA construct will preferably comprise 5 different promoters to prevent deletion of units by recombination. Preferably at least one of the promoters is a promoter of the invention.

Alternatively, the coding sequence may code for the expression of an antisense RNA and/or an RNAi (RNA interference) construct. An example of expressing an antisense-RNA is shown in Appl Environ Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52.) Complete inactivation of the expression of a gene is useful for instance for the inactivation of genes controlling undesired side branches of metabolic pathways, for instance to increase the production of specific secondary metabolites such as (beta-lactam) antibiotics or carotenoids. Complete inactivation is also useful to reduce the production of toxic or unwanted compounds (chrysogenin in Penicillium; Aflatoxin in Aspergillus: MacDonald K D et al: heterokaryon studies and the genetic control of penicillin and chrysogenin production in Penicillium chrysogenum. J Gen Microbiol. (1963) 33:375-83). Complete inactivation is also useful to alter the morphology of the organism in such a way that the fermentation process and down stream processing is improved.

Another embodiment of the invention relates to the extensive metabolic reprogramming or engineering of a host cell. Introduction of complete new pathways and/or modification of unwanted pathways will provide a cell specifically adapted for the production of a specific biological compound such as a protein or a metabolite.

In the methods of the present invention, when the coding sequence codes for a polypeptide, said polypeptide may also include a fused or hybrid polypeptide in which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide. Techniques for producing fusion polypeptides are known in the art, and include, ligating the coding sequences encoding the polypeptides so that they are in frame and expression of the fused polypeptide is under control of the same promoter(s) and terminator. The hybrid polypeptide may comprise a combination of partial or complete polypeptide sequences obtained from at least two different polypeptides wherein one or more may be heterologous to the fungal cell.

The DNA construct may comprise one or more control sequences in addition to the promoter DNA sequence, which direct the expression of the coding sequence in a suitable host cell under conditions compatible with the control sequences. Expression will be understood to include any step involved in the production of the polypeptide including, but not limited to, transcription, post-transcriptional modification, translation, post-translational modification, and secretion. One or more control sequences may be native to the coding sequence or to the host. Alternatively, one or more control sequences may be replaced with one or more control sequences foreign to the nucleic acid sequence for improving expression of the coding sequence in a host cell.

“DNA construct” is defined herein as a nucleic acid molecule, either single or double-stranded, which is isolated from a naturally occurring gene or which has been modified to contain segments of nucleic acid combined and juxtaposed in a manner that would not otherwise exist in nature. The term DNA construct is synonymous with the term expression cassette when the DNA construct contains a coding sequence and all the control sequences required for expression of the coding sequence.

The term “control sequences” is defined herein to include all components, which are necessary or advantageous for the expression of a coding sequence, including the promoter of the invention. Each control sequence may be native or foreign to the nucleic acid sequence encoding the polypeptide. Such control sequences include, but are not limited to, a leader, a translational initiator sequence (as described in Kozak, 1991, J. Biol. Chem. 266:19867-19870), a translational initiator coding sequence, a polyadenylation sequence, a propeptide sequence, a signal peptide sequence, an upstream activating sequence, the promoter of the invention including variants, fragments, and hybrid and tandem promoters derived thereof, a transcription terminator, and a translational terminator. At a minimum, the control sequences include transcriptional and translational stop signals and (part of) the promoter of the invention. The control sequences may be provided with linkers for the purpose of introducing specific restriction sites facilitating ligation of the control sequences with the coding region of the nucleic acid sequence encoding a polypeptide.

The control sequence may be a suitable transcription terminator sequence, i.e. a sequence recognized by a host cell to terminate transcription. The terminator sequence is in operative association with the 3′ terminus of the coding sequence encoding the polypeptide. Any terminator, which is functional in the host cell of choice, may be used in the present invention.

Preferred terminators for filamentous fungal host cells are obtained from the genes for A. oryzae TAKA amylase, A. niger glucoamylase, A. nidulans anthranilate synthase, A. niger alpha-glucosidase, trpC gene, and Fusarium oxysporum trypsin-like protease.

The control sequence may also be a suitable leader sequence, i.e. a 5′ nontranslated region of a mRNA which is important for translation by the host cell. The leader sequence is in operative association with the 5′ terminus of the nucleic acid sequence encoding the polypeptide. Any leader sequence that is functional in the host cell of choice may be used in the present invention.

Preferred leaders for filamentous fungal host cells are obtained from the genes for A. oryzae TAKA amylase, A. nidulans triose phosphateisomerase and A. niger glaA.

The control sequence may also be a polyadenylation sequence, a sequence in operative association with the 3′ terminus of the nucleic acid sequence and which, when transcribed, is recognized by the host cell as a signal to add polyadenosine residues to transcribed mRNA. Any polyadenylation sequence, which is functional in the host cell of choice may be used in the present invention.

Preferred polyadenylation sequences for filamentous fungal host cells are obtained from the genes for A. oryzae TAKA amylase, A. niger glucoamylase, A. nidulans anthranilate synthase, Fusarium oxysporum trypsin-like protease, and A. niger alpha-glucosidase.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for filamentous fungal host cells are the signal peptide coding regions obtained from the genes for A. oryzae TAKA amylase, A. niger neutral amylase, A. ficuum phytase, A. niger glucoamylase, A. niger endoxylanase, Rhizomucor miehei aspartic proteinase, Humicola insolens cellulase, and Humicola lanuginosa lipase.

Useful signal peptides for yeast host cells are obtained from the genes for Saccharomyces cerevisiae alpha-factor and Saccharomyces cerevisiae invertase. Other useful signal peptide coding regions are described by Romanos et al., 1992, supra.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, Myceliophthora thermophila laccase (WO 95/33836) and A. niger endoxylanase (endo1).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, and trp operator systems. In yeast, the ADH2 system or GAL1 system may be used. In filamentous fungi, the TAKA alpha-amylase promoter, A. niger glucoamylase promoter, A. oryzae glucoamylase promoter, A. tubingensis endoxylanase (xlnA) promoter, A. niger nitrate reductase (niaD) promoter, Trichoderma reesei cellobiohydrolase promoter and the A. nidulans alcohol and aldehyde dehydrogenase (alcA and aldA, respectively) promoters as described in U.S. Pat. No. 5,503,991) may be used as regulatory sequences. Other examples of regulatory sequences are those, which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the polypeptide would be in operative association with the regulatory sequence.

Important can be removal of creA binding sites (carbon catabolite repression as described earlier in EP 673 429), change of pacC and areA (for pH and nitrogen regulation).

Preferably, the DNA construct comprises a promoter DNA sequence from the invention, a coding sequence in operative association with said promoter DNA sequence and translational control sequences such as:
one translational termination sequence orientated in 5′ towards 3′ direction selected from the following list of sequences: TAAG, TAGA and TAAA, preferably TAAA, and/or
one translational initiator coding sequence orientated in 5′ towards 3′ direction selected from the following list of sequences: GCTACCCCC; GCTACCTCC; GCTACCCTC; GCTACCTTC; GCTCCCCCC; GCTCCCTCC; GCTCCCCTC; GCTCCCTTC; GCTGCCCCC; GCTGCCTCC; GCTGCCCTC; GCTGCCTTC; GCTTCCCCC; GCTTCCTCC; GCTTCCCTC; and GCTTCCTTC, preferably GCT TCC TTC, and/or
one transcriptional initiator sequence selected from the following list of sequences: 5′-mwChkyCAAA-3′; 5′-mwChkyCACA-3′ or 5′-mwChkyCAAG-3′, using ambiguity codes for nucleotides: m (A/C); w (A/T); y (C/T); k (G/T); h (A/C/T), preferably 5′-CACCGTCAAA-3′ or 5′-CGCAGTCAAG-3′.

In the context of this invention, the term “translational initiator coding sequence” is defined as the nine nucleotides immediately downstream of the initiator or start codon of the open reading frame of a DNA coding sequence. The initiator or start codon encodes for the AA methionine. The initiator codon is typically ATG, but may also be any functional start codon such as GTG.

In the context of this invention, the term “translational termination sequence” is defined as the three or four nucleotides starting from the translational stop codon at the 3′ end of the open reading frame or nucleotide coding sequence and oriented in 5′ towards 3′ direction.

In the context of this invention, the term “translational initiator sequence” is defined as the ten nucleotides immediately upstream of the initiator or start codon of the open reading frame of a DNA sequence coding for a polypeptide. The initiator or start codon encodes for the AA methionine. The initiator codon is typically ATG, but may also be any functional start codon such as GTG. It is well known in the art that uracil, U, replaces the deoxynucleotide thymine, T, in RNA.

The present invention also relates to recombinant expression vectors comprising a promoter of the present invention, a coding sequence encoding a polypeptide, and transcriptional and translational initiator and stop signals.

The various coding and control sequences described above may be joined together to produce a recombinant expression vector which may include one or more convenient restriction sites to allow for insertion or substitution of the promoter and/or coding sequence encoding the polypeptide at such sites. Alternatively, fusion of coding sequence and promoter can be done by e.g. sequence overlap extension using PCR (SOE-PCR), as described in Gene. 1989 Apr. 15; 77(1):51-9. Ho S N, Hunt H D, Horton R M, Pullen J K, Pease L R “Site-directed mutagenesis by overlap extension using the polymerase chain reaction”) or by cloning using the Gateway™ cloning system (Invitrogen). Alternatively, the coding sequence may be expressed by inserting the coding sequence or a DNA construct comprising the promoter and/or coding sequence into an appropriate vector for expression. In creating the expression vector, the coding sequence is located in the vector so that the coding sequence is in operative association with a promoter of the present invention and one or more appropriate control sequences for expression.

The recombinant expression vector may be any vector (e.g., a plasmid or virus), which can be conveniently subjected to recombinant DNA procedures and can effectuate expression of the coding sequence. The choice of the vector will typically depend on the compatibility of the vector with the host cell into which the vector is to be introduced. The vectors may be linear or closed circular plasmids.

The vector may be an autonomously replicating vector, i.e., a vector, which exists as an extrachromosomal entity, the replication of which is independent of chromosomal replication, e.g. a plasmid, an extrachromosomal element, a minichromosome, or an artificial chromosome. For autonomous replication, the vector may comprise an origin of replication enabling the vector to replicate autonomously in the host cell in question. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (see, e.g., Ehrlich, 1978, Proceedings of the National Academy of Sciences USA 75:1433). An example of an autonomously maintained cloning vector in a filamentous fungus is a cloning vector comprising the AMA1-sequence. AMA1 is a 6.0-kb genomic DNA fragment isolated from A. nidulans, which is capable of Autonomous Maintenance in Aspergillus (see e.g. Aleksenko and Clutterbuck (1997), Fungal Genet. Biol. 21: 373-397).

Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers, which permit easy selection of transformed cells. The host may be co-transformed with at least two vectors, one comprising the selection marker. A selectable marker is a gene the product of which provides for biocide or viral resistance, resistance to heavy metals, prototrophy to auxotrophs, and the like. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof. Marker conferring resistance against e.g. phleomycin, hygromycin B or G418 can also be used. Preferred for use in a Rasamsonia cell are the ble and hygB selection markers.

For integration into the host cell genome, the vector may rely on the promoter sequence and/or coding sequence encoding the polypeptide or any other element of the vector for stable integration of the vector into the genome by homologous or non-homologous recombination. Alternatively, the vector may contain additional nucleic acid sequences for directing integration by homologous recombination into the genome of the host cell. The additional nucleic acid sequences enable the vector to be integrated into the host cell genome at a predetermined target location(s) in the chromosome(s). To increase the likelihood of integration at a precise location, the integration elements should preferably contain a sufficient number of nucleic acids, such as 30 to 1,500 base pairs, preferably 100 to 1,500 base pairs, more preferably 400 to 1,500 base pairs, more preferably 800 to 1,500 base pairs, and most preferably at least 2 kb, which are highly homologous with the corresponding target sequence to enhance the probability of homologous recombination. The integration elements may be any sequence that is homologous with the target sequence in the genome of the host cell. Furthermore, the integration elements may be non-encoding or encoding nucleic acid sequences. In order to promote targeted integration, the cloning vector is preferably linearized prior to transformation of the host cell. Linearization is preferably performed such that at least one but preferably either end of the cloning vector is flanked by sequences homologous to the target locus.

Preferably, the integration elements in the cloning vector, which are homologous to the target locus are derived from a highly expressed locus meaning that they are derived from a gene, which is capable of high expression level in the fungal host cell. A gene capable of high expression level, i.e. a highly expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1). A number of preferred highly expressed fungal genes are given by way of example: the amylase, glucoamylase, alcohol dehydrogenase, xylanase, glyceraldehyde-phosphate dehydrogenase or cellobiohydrolase genes from Aspergilli or Trichoderma.

On the other hand, the vector may be integrated into the genome of the host cell by non-homologous recombination.

More than one copy of a nucleic acid sequence encoding a biological compound may be inserted into the host cell to increase production of the gene product. This can be done, preferably by integrating into its genome copies of the DNA sequence, more preferably by targeting the integration of the DNA sequence at a highly expressed locus. Alternatively, this can be done by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

The present invention also relates to recombinant host cells, comprising a promoter DNA sequence of the present invention in operative association with a coding sequence, said host cell being advantageously used in the production of a biological compound. A vector comprising a promoter of the present invention in operative association with a coding sequence, is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the origin of the coding sequence and to the origin of the promoter of the invention. The skilled person would know how to choose the best suited host cell.

The present invention also relates to recombinant host cells, comprising more than one promoter DNA sequence of the present invention, each promoter preferably being in operative association with a coding sequence. Such host cells may be advantageously used in the recombinant production of at least one biological compound. Alternatively, the recombinant host cells of the present invention may comprise one or more promoters of the present invention in combination with promoters known in the art. Such promoters known in the art include, but are not limited to: the promoters obtained from the genes for A. tubigensis xlnA, A. oryzae TAKA amylase, Rhizomucor miehei aspartic proteinase, A. niger neutral alpha-amylase, A. niger acid stable alpha-amylase, A. niger or A. awamori glucoamylase (glaA), A. niger or A. awamori endoxylanase (xlnA) or beta-xylosidase (xlnD), T. reesei cellobiohydrolase I (CBHI), R. miehei lipase, A. oryzae alkaline protease, A. oryzae triose phosphate isomerase, A. nidulans acetamidase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the polynucleotides encoding A. niger neutral alpha-amylase and A. oryzae triose phosphate isomerase), and mutant, truncated, and hybrid promoters thereof. Other examples of promoters are the promoters described in WO2006/092396 and WO2005/100573, which are herein incorporated by reference. An even other example of the use of promoters is described in WO2008/098933. Examples of inducible (heterologous) promoters are the alcohol inducible promoter alcA, the tet system using the tetracycline-responsive promoter, the estrogen-responsive promoter (Pachlinger et al. (2005), Appl & Environmental Microbiol 672-678).

The host cell of the present invention and the host cell used in the methodology of the present invention may be any host cell. Preferably, the host cell of the present invention is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

Preferably, the filamentous fungal host cell is a cell of a genus of Acremonium, Agaricus, Aspergillus, Aureobasidium, Chrysosporium, Coprinus, Cryptococcus, Filobasidium, Fusarium, Geosmithia, Humicola, Magnaporthe, Mucor, Myceliophthora, Neocallimastix, Neurospora, Paecilomyces, Penicillium, Piromyces, Panerochaete, Pleurotus, Rasamsonia, Schizophyllum, Talaromyces, Thermoascus, Thermomyces, Thielavia, Tolypocladium, and Trichoderma

In a more preferred embodiment, the filamentous fungal host cell is an Humicola grisea var. thermoidea, Humicola lanuginosa, Myceliophthora thermophila, Papulaspora thermophilia, Rasamsonia byssochlamydoides, Rasamsonia emersonii, Rasamsonia argillacea, Rasamsonia eburnean, Rasamsonia brevistipitata, Rasamsonia cylindrospora, Rhizomucor pusillus, Rhizomucor miehei, Talaromyces bacillisporus, Talaromyces leycettanus, Talaromyces thermophilus, Thermomyces lenuginosus Thermoascus crustaceus, Thermoascus thermophilus Thermoascus aurantiacus or Thielavia terrestris cell. In another more preferred embodiment, the filamentous fungal host cell is a Aspergillus awamori, Aspergillus foetidus, Aspergillus japonicus, A. nidulans, A. niger, A. sojae, A. oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatun, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another more preferred embodiment, the filamentous fungal host cell is a, Mucor miehei, Myceliophthora thermophila, Neurospora crassa, Penicillium purpurogenum, Penicillium chrysogenum, Trichoderma harzianum, Trichoderma koningii, Trichoderma longibrachiatum, Trichoderma reesei, or Trichoderma viride cell. In a most preferred embodiment, the filamentous fungal host cell is a species selected from the group consisting of Rasamsonia emersonii, Aspergillus niger, Aspergillus oryzae, Aspergillus sojae, Myceliophthora thermophila, Trichoderma reesei or Penicillium chrysogenum. A most preferred Rasamsonia emersonii host cell is CBS393.64 or derivatives thereof.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL) Rasamsonia. emersonii ATCC16479, Aspergillus niger CBS 513.88, Aspergillus oryzae ATCC 20423, IFO 4177, ATCC 1011, ATCC 9576, ATCC14488-14491, ATCC 11601, ATCC12892, P. chrysogenum CBS 455.95, Penicillium citrinum ATCC 38065, Penicillium chrysogenum P2, Acremonium chrysogenum ATCC 36225 or ATCC 48272, Trichoderma reesei ATCC 26921 or ATCC 56765 or ATCC 26921, Aspergillus sojae ATCC11906, Chrysosporium lucknowense ATCC44006.

The host cell may be a wild type filamentous fungus host cell or a variant, a mutant or a genetically modified filamentous fungus host cell.

Fungal cells may be transformed by a process involving protoplast formation, transformation of the protoplasts, and regeneration of the cell wall in a manner known per se. A suitable procedures for transformation of Rasamsonia host cells is described in WO2011\054899. Suitable procedures for transformation of Aspergillus host cells are described in EP 238 023 and Yelton et al., 1984, Proceedings of the National Academy of Sciences USA 81: 1470-1474. Suitable procedures for transformation of Aspergillus and other filamentous fungal host cells using Agrobacterium tumefaciens are described in e.g. Nat Biotechnol. 1998 September; 16(9):839-42. Erratum in: Nat Biotechnol 1998 November; 16(11):1074. Agrobacterium tumefaciens-mediated transformation of filamentous fungi. de Groot M J, Bundock P, Hooykaas P J, Beijersbergen A G. Unilever Research Laboratory Vlaardingen, The Netherlands. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

The “biological compound” may be any biopolymer or metabolite. The biological compound may be encoded by a single coding sequence or a series of coding sequences composing a biosynthetic or metabolic pathway or may be the direct result of the product of a single coding sequence or products of a series of coding sequences. The biological compound may be native to the host cell or heterologous.

The term “heterologous biological compound” is defined herein as a biological compound which is not native to a given host cell or a native biological compound in which structural modifications have been made to alter the native biological compound.

The term “biopolymer” is defined herein as a chain (or polymer) of identical, similar, or dissimilar subunits (monomers). The biopolymer may be any biopolymer. The biopolymer may for example be, but is not limited to, a nucleic acid like RNA, polyamine, polyol, polypeptide (or polyamide), or polysaccharide.

According to a preferred embodiment, the biological compound produced is a polypeptide. According to a more preferred embodiment, the polypeptide produced is encoded by the coding sequence present in the DNA construct, said DNA construct comprising the promoter of the invention operably linked to said coding sequence. The polypeptide may be any polypeptide having a biological activity of interest. The term “polypeptide” is not meant herein to refer to a specific length of the encoded product and, therefore, encompasses peptides, oligopeptides, and proteins. The term “polypeptide” also encompasses two or more polypeptides combined to form the encoded product.

Polypeptides also include hybrid polypeptides, which comprise a combination of partial or complete polypeptide sequences obtained from at least two different polypeptides wherein one or more may be heterologous to the host cell. Polypeptides further include naturally occurring allelic and engineered variations of the above-mentioned polypeptides and hybrid polypeptides.

The polypeptide may be native or heterologous to a given host cell. The term “heterologous polypeptide” is defined herein as a polypeptide, which is not native to a given host cell. Alternatively an heterologous polypeptide is a native polypeptide in which modifications have been made to alter the native sequence, or a native polypeptide whose expression is quantitatively altered as a result of a manipulation of the fungal cell by recombinant DNA techniques. For example, a native polypeptide may be recombinantly produced by, e.g., placing the sequence encoding the polypeptide under the control of the promoter of the present invention to enhance expression of the polypeptide, to expedite export of a native polypeptide of interest outside the cell by use of a signal sequence, and to increase the copy number of a gene encoding the polypeptide normally produced by the cell.

The polypeptide may be a collagen or gelatin, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase. The polypeptide may be an enzyme secreted extracellularly. Such enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an aminopeptidase, amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, proteolytic enzyme, invertase, laccase, lipase, mannosidase, mutanase, oxidase, pectinolytic enzyme, peroxidase, phospholipase, polyphenoloxidase, ribonuclease, transglutaminase, or glucose oxidase, hexose oxidase, monooxygenase.

Alternatively, the coding sequence, operably linked to a promoter of the present invention may encode an intracellular protein such as for example a chaperone or transcription factor. An example of this is described in Appl Microbiol Biotechnol. 1998 October; 50(4):447-54 (“Analysis of the role of the gene bipA, encoding the major endoplasmic reticulum chaperone protein in the secretion of homologous and heterologous proteins in black Aspergilli. Punt P J, van Gemeren I A, Drint-Kuijvenhoven J, Hessing J G, van Muijlwijk-Harteveld G M, Beijersbergen A, Verrips C T, van den Hondel C A). This can be used for example to improve the efficiency of a host cell as protein producer or as metabolite if this coding sequence, such as a chaperone or transcription factor, was known to be a limiting factor in protein or metabolite production.

The biological compound may be a polysaccharide. The polysaccharide may be any polysaccharide, including, but not limited to, a mucopolysaccharide (e.g. heparin and hyaluronic acid) and nitrogen-containing polysaccharide (eg. chitin). In a more preferred option, the polysaccharide is hyaluronic acid.

Alternatively, the biological compound may be a metabolite. The term “metabolite” encompasses both primary and secondary metabolites; the metabolite may be any metabolite. A preferred metabolite is citric acid.

According to another preferred embodiment, the biological compound produced is a metabolite. According to a more preferred embodiment, the coding sequence present in the DNA construct encodes an enzyme involved in the production of a metabolite, said DNA construct comprising the promoter of the invention operably linked to said coding sequence.

Alternatively, several coding sequences may be present in the DNA construct of the present invention. Each coding sequence may encode a distinct enzyme involved in a metabolic or biosynthetic pathway leading to the production of a metabolite. Primary metabolites are products of primary or general metabolism of a cell, which are concerned with energy metabolism, growth, and structure. Secondary metabolites are products of secondary metabolism (see, for example, R. B. Herbert, The Biosynthesis of Secondary Metabolites, Chapman and Hall, New York, 1981).

The primary metabolite may be, but is not limited to, an amino acid, fatty acid, nucleoside, nucleotide, sugar, triglyceride, or vitamin. A preferred primary metabolite is citric acid.

The secondary metabolite may be, but is not limited to, an alkaloid, coumarin, flavonoid, polyketide, quinine, steroid, peptide, or terpene. The secondary metabolite may be an antibiotic, antifeedant, attractant, bacteriocide, fungicide, hormone, insecticide, or rodenticide. Preferred antibiotics are cephalosporins and beta-lactams.

The biological compound may also be a selectable marker. A selectable marker is product, which provides resistance against a biocide or virus, resistance to heavy metals, prototrophy to auxotrophs, and the like. Selectable markers include, but are not limited to, amdS (acetamidase), arg B (ornithinecarbamoyltransf erase), bar (phosphinothricinacetyltransf erase), hygB (hygromycin phosphotransferase), niaD (nitratereductase), pyrG (orotidine-5′-phosphate decarboxylase), sC (sulfate adenyltransferase), trpC (anthranilate synthase), ble (phleomycin resistance protein), as well as equivalents thereof.

In the production methods of the present invention, the cells are cultivated in a nutrient medium suitable for production of the biological compound which may be, but is not limited to, a polypeptide or metabolite using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the coding sequence to be expressed and/or the biological compound to be isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the biological compound is secreted into the nutrient medium, the biological compound can be recovered directly from the medium. If the biological compound, which may be, but is not limited to, a polypeptide or metabolite is not secreted, it can be recovered from cell lysates.

The resulting biological compound, which may be, but is not limited to, a polypeptide or metabolite may be recovered by methods known in the art. For example, a polypeptide or metabolite may be recovered from the nutrient medium by conventional procedures including, but not limited to, centrifugation, filtration, extraction, spray-drying, evaporation, or precipitation.

Polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989). Polypeptides may be detected using methods known in the art that are specific for the polypeptides. These detection methods may include use of specific antibodies, formation of an enzyme product, or disappearance of an enzyme substrate.

The present invention also relates to DNA constructs for altering the expression of a coding sequence encoding a polypeptide, which is endogenous to a fungal host cell. The constructs may contain the minimal number of components necessary for altering expression of the endogenous gene.

In one embodiment, the nucleic acid constructs preferably contain (a) a targeting sequence, (b) a promoter DNA sequence of the present invention, (c) an exon, and (d) a splice-donor site. Upon introduction of the nucleic acid construct into a cell, the construct integrates by homologous recombination into the cellular genome at the endogenous gene site. The targeting sequence directs the integration of elements (a)-(d) into the endogenous gene such that elements (b)-(d) are in operative association with the endogenous gene.

In another embodiment, the nucleic acid constructs contain (a) a targeting sequence, (b) a promoter DNA sequence of the present invention, (c) an exon, (d) a splice-donor site, (e) an intron, and (f) a splice-acceptor site, wherein the targeting sequence directs the integration of elements (a)-(f) such that elements (b)-(f) are in operative association with the endogenous gene. However, the constructs may contain additional components such as a selectable marker. The selectable markers that can be used were earlier described.

In both embodiments, the introduction of these components results in production of a new transcription unit in which expression of the endogenous gene is altered. In essence, the new transcription unit is a fusion product of the sequences introduced by the targeting constructs and the endogenous gene. In one embodiment in which the endogenous gene is altered, the gene is activated. In this embodiment, homologous recombination is used to replace, disrupt, or disable the regulatory region normally associated with the endogenous gene of a parent cell through the insertion of a regulatory sequence, which causes the gene to be expressed at higher levels than evident in the corresponding parent cell.

The targeting sequence can be within the endogenous gene, immediately adjacent to the gene, within an upstream gene, or upstream of and at a distance from the endogenous gene. One or more targeting sequences can be used. For example, a circular plasmid or DNA fragment preferably employs a single targeting sequence, while a linear plasmid or DNA fragment preferably employs two targeting sequences.

The constructs further contain one or more exons of the endogenous gene. An exon is defined as a DNA sequence, which is copied into RNA and is present in a mature mRNA molecule such that the exon sequence is in-frame with the coding region of the endogenous gene. The exons can, optionally, contain DNA, which encodes one or more amino acids and/or partially encodes an amino acid. Alternatively, the exon contains DNA which corresponds to a 5′ non-encoding region. Where the exogenous exon or exons encode one or more amino acids and/or a portion of an amino acid, the nucleic acid construct is designed such that, upon transcription and splicing, the reading frame is in-frame with the coding region of the endogenous gene so that the appropriate reading frame of the portion of the mRNA derived from the second exon is unchanged. The splice-donor site of the constructs directs the splicing of one exon to another exon. Typically, the first exon lies 5′ of the second exon, and the splice-donor site overlapping and flanking the first exon on its 3′ side recognizes a splice-acceptor site flanking the second exon on the 5′ side of the second exon. A splice-acceptor site, like a splice-donor site, is a sequence, which directs the splicing of one exon to another exon. Acting in conjunction with a splice-donor site, the splicing apparatus uses a splice-acceptor site to effect the removal of an intron.

A preferred strategy for altering the expression of a given DNA sequence comprises the deletion of the given DNA sequence and/or replacement of the endogenous promoter sequence of the given DNA sequence by a modified promoter DNA sequence, such as a promoter of the invention.

Alternatively or in combination with other mentioned techniques, a technique based on in vivo recombination of cosmids in E. coli can be used, as described in: A rapid method for efficient gene replacement in the filamentous fungus A. nidulans (2000) Chaveroche, M-K., Ghico, J-M. and d'Enfert C; Nucleic acids Research, vol 28, no 22. This technique is applicable to other filamentous fungi like for example R. emersonii.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The present invention is further described by the following examples, which should not be construed as limiting the scope of the invention.

### EXAMPLES

It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Experimental Information

### Strains

The Rasamsonia emersonii (R. emersonii) strains used herein are derived from ATCC16479, which is used as wild-type strain. ATCC16479 was formerly also known as Talaromyces emersonii and Penicillium geosmithia emersonii. Upon the use of the name Rasamsonia emersonii also Talaromyces emersonii is meant. Other strain designations of R. emersonii ATCC16479 are CBS393.64, IF031232 and IM1116815.

Rasamsonia (Talaromyces) emersonii strain TEC-142 is deposited at CENTRAAL BUREAU VOOR SCHIMMELCULTURES, Uppsalalaan 8, P.O. Box 85167, NL-3508 AD Utrecht, The Netherlands on 1st July 2009 having the Accession Number CBS 124902. TEC-142S is a single isolate of TEC-142.

### Molecular Biology Techniques

In these strains, using molecular biology techniques known to the skilled person (see: Sambrook & Russell, Molecular Cloning: A Laboratory Manual, 3rd Ed., CSHL Press, Cold Spring Harbor, N.Y., 2001), several genes were over expressed and others were down regulated as described below. Examples of the general design of expression vectors for gene over expression and disruption vectors for down-regulation, transformation, use of markers and selective media can be found in for example WO199846772, WO199932617, WO2001121779, WO2005095624, EP 635574B and WO2005100573.

Media and solutions

Potato dextrose agar, PDA, (Fluka, Cat. No. 70139)

| | |
|---|---|
| Potato extract | 4 g/l |
| Dextrose | 20 g/l |
| Bacto agar | 15 g/l |
| pH | 5.4 |
| Water | Adjust to one liter |
| Sterilize | 20 min at 120° C. |

Rasamsonia agar medium

| | |
|---|---|
| Salt fraction no. 3 | 15 g |
| Cellulose (3%) | 30 g |
| Bacto peptone | 7.5 g |
| Grain flour | 15 g |
| KH₂PO₄ | 5 g |
| CaCl2•2aq | 1 g |
| Bacto agar | 20 g |
| pH | 6.0 |
| Water | Adjust to one liter |
| Sterilize | 20 min at 120° C. |

### Salt Fraction Composition

The “salt fraction no. 3” was fitting the disclosure of WO98/37179, Table 1. Deviations from the composition of this table were CaCl2.2aq 1.0 g/l, KCl 1.8 g/L, citric acid 1 aq 0.45 g/L (chelating agent).

### Shake Flask Media for Rasamsonia

Rasamsonia medium 1

| | | |
|---|---|---|
| Glucose | 20 | g/L |
| Yeast extract (Difco) | 20 | g/L |
| Clerol FBA3107 (AF) | 4 | drops/L |

| | |
|---|---|
| pH | 6.0 |
| Sterilize | 20 min at 120° C. |

Rasamsonia medium 2

| | | |
|---|---|---|
| Salt fraction no. 3 | 15 | g |
| Cellulose | 20 | g |
| Bacto peptone | 4 | g |
| Grain flour | 7.5 | g |
| KH₂PO₄ | 10 | g |
| CaCl₂•2H20 | 0.5 | g |
| Clerol FBA3107 (AF) | 0.4 | ml |

| | |
|---|---|
| pH | 5 |
| Water | Adjust to one liter |
| Sterilize | 20 min at 120° C. |

Rasamsonia medium 3

| | | |
|---|---|---|
| Salt fraction no. 3 | 15 | g |
| Glucose | 24 | g |
| Grain flour | 7.5 | g |
| KH₂PO₄ | 10 | g |
| CaCl₂•2H20 | 0.5 | g |
| Clerol FBA3107 (AF) | 0.4 | ml |

| | |
|---|---|
| pH | 5 |
| Water | Adjust to one liter |
| Sterilize | 20 min at 120° C. |

### Spore Batch Preparation for Rasamsonia

Strains were grown from stocks on Rasamsonia agar medium in 10 cm diameter Petri dishes for 5-7 days at 40° C. For MTP fermentations, strains were grown in 96-well plates containing Rasamsonia agar medium. Strain stocks were stored at −80° C. in 10% glycerol.

### Chromosomal DNA Isolation

Strains were grown in YGG medium (per liter: 8 g KCl, 16 g glucose.H₂O, 20 ml of 10% yeast extract, 10 ml of 100× pen/strep, 6.66 g YNB+amino acids, 1.5 g citric acid, and 6 g K₂HPO₄). for 16 hours at 42° C., 250 rpm, and chromosomal DNA was isolated using the DNeasy plant mini kit (Qiagen, Hilden, Germany).

### Protein Analysis

Proteins in 65 μl of supernatant were precipitated by adding 228 μl TCA-aceton (1.2 g trichloric acid, 9 ml of acetone, 1 ml of H₂O. After precipitating for 3 hours at −20° C., samples were centrifuged at 14.000 rpm at 4° C. for 10 min in an eppendorf centrifuge and pellets were washed with acetone. Dried pellets were dissolved in 1× sample buffer (25 μl of LDS sample buffer (Invitrogen, Breda, The Netherlands), 10 μl of reducing agent (Invitrogen, Breda, The Netherlands), 65 1 of H₂O).

Protein samples were separated under reducing conditions on NuPAGE 4-12% Bis-Tris gel (Invitrogen, Breda, The Netherlands) and stained as indicated. Gels were stained with either InstantBlue (Expedeon, Cambridge, United Kingdom), SimplyBlue safestain (Invitrogen, Breda, The Netherlands) or Sypro Ruby (Invitrogen, Breda, The Netherlands)) according to manufacturer's instructions.

For Western blotting, proteins were transferred to nitrocellulose. The nitrocellulose filter was blocked with TBST (Tris buffered saline containing 0.1% Tween 40) containing 3% skim-milk and incubated for 16 hours with anti-FLAG M2 antibody (Sigma, Zwijndrecht, The Netherlands). Blots were washed twice with TBST for 10 minutes and stained with Horse-radish-peroxidase conjugated rabbit-anti-mouse antibody (DAKO, Glostrup, Denmark) for 1 hour. After washing the blots five times with TBST for 10 minutes, proteins were visualized using SuperSignal (Pierce, Rockford, U.S.A). Optionally, the Western blot can be quantified using a ChemiDoc System (Biorad, Veenendaal, The Netherlands).

### Example 1

### Construction of a DNA Construct Comprising a Promoter of the Invention in Operative Association with a Coding Sequence

This example describes the construction of an expression construct comprising a promoter of the invention in operative association with a coding sequence. The coding sequence or reporter construct used here is the FLAG-tagged R. emersonii endoglucanase gene (EBA7-FLAG). EBA7-FLAG is used as the reporter enzyme to be able to detect the recombinant protein by Western blotting using a FLAG-tag specific antibody. The constructs were randomly integrated into the genome.

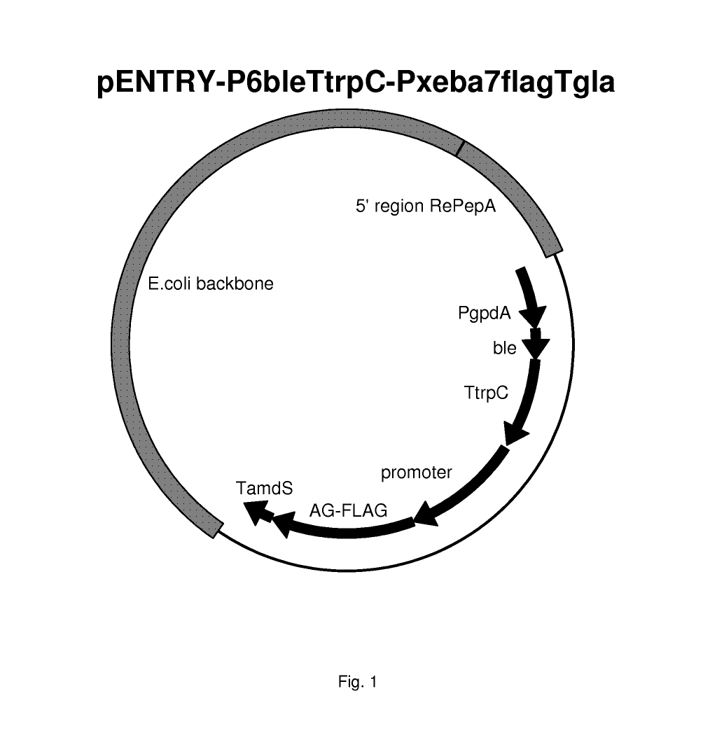

Vector pENTRY-P6bleTtrpC-Pxeba7flagTgla was constructed according to routine cloning procedures. The vector comprises a ble expression cassette consisting of the A. nidulans gpdA promoter (P6), ble coding region (ble) and A. nidulans TrpC terminator (TtrpC), a promoter of interest (Px), the EBA7-FLAG reporter coding region (eba7flag) and the A. niger glucoamylase terminator (FIG. 1). Five R. emersonii promoters were cloned into the vector: R. emersonii cellobiohydrolase-I (PcbhI, SEQ ID NO: 1), R. emersonii acetyl xylan esterase (Pace, SEQ ID NO: 2), R. emersonii endoglucanase (Peg, SEQ ID NO: 3), R. emersonii cellobiohydrolase-II (PcbhII, SEQ ID NO: 4), and R. emersonii beta-glucosidase (Pbg, SEQ ID NO: 5). In addition, the A. nidulans gpdA promoter was cloned into the vector to compare the activity of the promoters with the A. nidulans gpdA promoter (PgpdA, SEQ ID NO: 6). The EBA7-FLAG reporter gene cassette was obtained from vector pGBFINEBA7, described in WO2011\054899. The 5 promoter constructs were tested for expression in R. emersonii.

### Example 2

### Expression of Promoter-Reporter Construct in Rasamsonia emersonii

The pENTRY-P6bleTtrpC-Pxeba7flagTgla promoter constructs described in Example 1 were used to transform R. emersonii strain TEC-142S using method as described earlier in WO2011\054899. Transformants were selected on phleomycin media and colony purified, and tested according to procedures as described in WO2011\054899. Spore batches were generated of transformants containing promoter-reporter constructs.

Transformants were grown in shake flasks in Rasamsonia medium 1 at 45° C., 250 rpm in an incubator shaker for 24 hours and this pre-culture was used to inoculate Rasamsonia medium 2 containing cellulose as C-source (approximately 10% inoculation). Samples were taken after 40 hours and proteins were precipitated using TCA-aceton and separated on SDS-PAGE. Recombinant FLAG-tagged glucoamylase was detected by Western blotting using a FLAG-tag specific antibody.

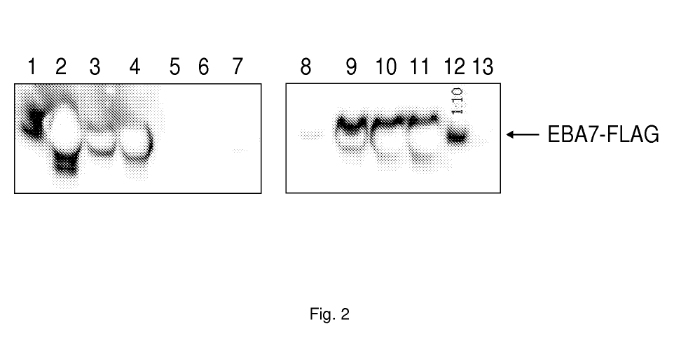

The result of the Western blot is shown in FIG. 2. Supernatants of transformants of all 6 promoter-reporter constructs showed a specific EBA7-FLAG protein band on Western blot. All of the 5 R. emersonii (hemi)cellulose promoters were able to drive expression of the AG-FLAG reporter gene and showed stronger expression compared to the A. nidulans gpdA promoter in a medium containing 2% cellulose under the tested condition (compare lanes 1-5 and 8-12 with lane 7).

### Example 3

### Construction of a DNA Construct Comprising a Promoter of the Invention in Operative Association with a Coding Sequence that is Targeted to the Genome

This example describes the construction of an expression construct comprising a promoter of the invention in operative association with a coding sequence. The coding sequence or reporter construct used here is the FLAG-tagged R. emersonii glucoamylase gene. Glucoamylase-FLAG is used as the reporter enzyme to be able to measure the activity of the promoter of the invention by glucoamylase activity measurements or by Western blotting using a FLAG-tag specific antibody. The promoter-reporter expression cassette was targeted integrated into the pepA locus.

In order to target the promoter-reporter constructs into the pepA locus, expression vectors were cloned for targeting. Genomic DNA of Rasamsonia emersonii strain CBS393.64 was sequenced and analyzed. The gene with translated protein annotated as protease pepA was identified. Sequences of Rasamsonia emersonii pepA (RePepA), comprising the genomic sequence of the ORF and approximately 3000 bp of the 5′ region and 2500 bp of the 3′ flanking regions, cDNA and protein sequence, are shown in sequence listings 7, 8 and 9, respectively.

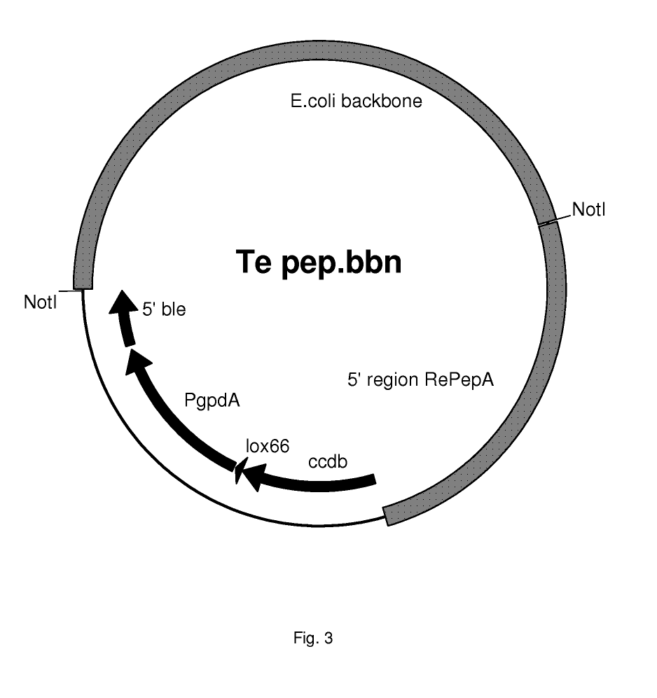

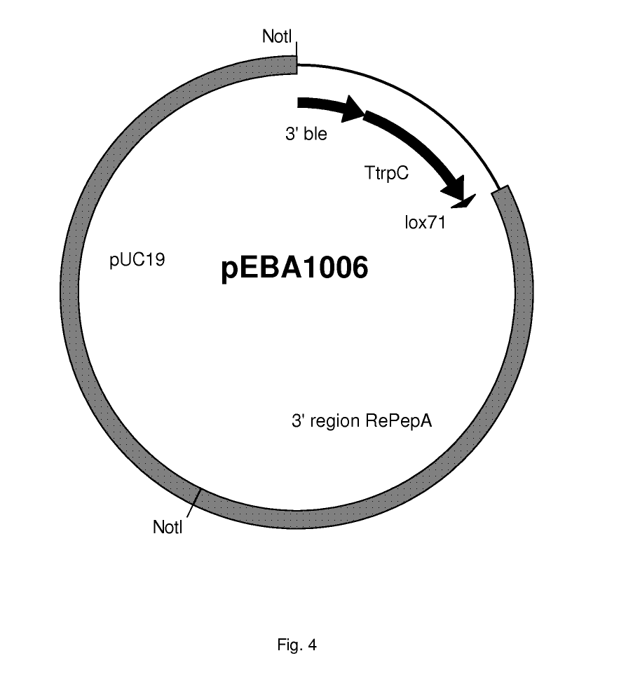

Two vectors were constructed according to routine cloning procedures for targeting into the RePepA locus. The insert fragments of both vectors together can be applied in the so-called “bipartite gene-targeting” method (Nielsen et al., 2006, 43: 54-64). This method is using two non-functional DNA fragments of a selection marker which are overlapping (see also WO2008113847 for further details of the bipartite method) together with gene-targeting sequences. Upon correct homologous recombination the selection marker becomes functional by integration at a homologous target locus. As also detailed in WO 2008113847, two different deletion vectors, Te pep.bbn and pEBA1006, were designed and constructed to be able to provide the two overlapping DNA molecules for bipartite gene-targeting. The first vector Te pep.bbn (General layout as in FIG. 3) comprises a 1500 bp 5′ flanking region approximately 1.5 kb upstream of the RePepA ORF for targeting in the RePepA locus (ORF and approximately 1500 bp of the RePepA promoter), a lox66 site, and the non-functional 5′ part of the ble coding region driven by the A. nidulans gpdA promoter (PgpdA-ble sequence missing the last 104 bases of the coding sequence at the 3′ end of ble, SEQ ID NO: 10). To allow efficient cloning of promoter-reporter cassettes in E. coli, a ccdB gene was inserted in between the 5′ RePepA flanking region and the lox66 site. The second pEBA1006 vector (General layout as in FIG. 4) comprises the non-functional 3′ part of the ble coding region and the A. nidulans trpC terminator (ble-TtrpC sequence missing the first 12 bases of the coding sequence at the 5′ end of ble, SEQ ID NO: 11), a lox71 site, and a 2500 bp 3′ flanking region of the RePepA ORF for targeting in the RePepA locus. Upon homologous recombination, the first and second non-functional fragments become functional producing a functional ble cassette. Both RePepA upstream and downstream gene flanking regions target for homologous recombination of the bipartite fragments at the predestined RePepA genomic locus.

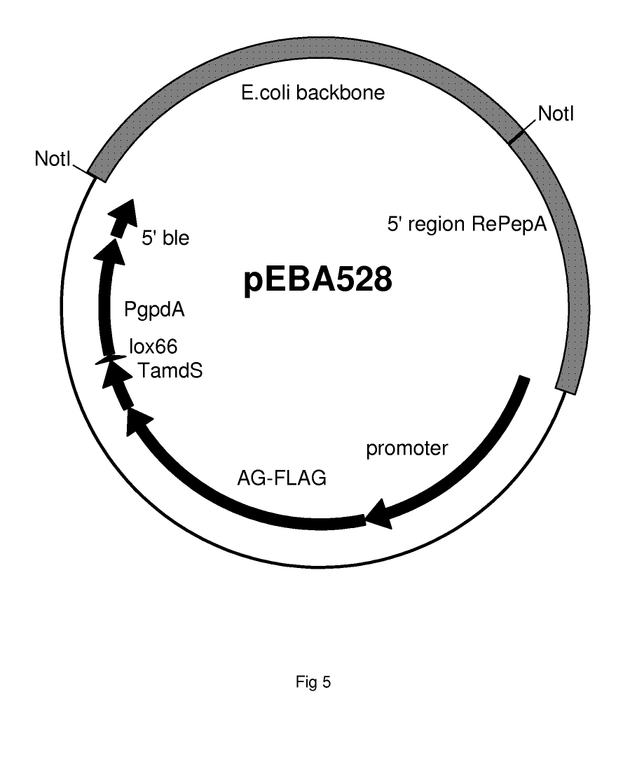

The ccdB gene in vector Te pep.bbn was replaced by promoter-reporter cassette according to routine cloning procedures. Six R. emersonii promoters, represented by SEQ ID NO 12, 13, 14, 15, 16 and 17, were cloned upstream of the FLAG-tagged R. emersonii glucoamylase coding region (AG-FLAG) with A. nidulans amdS terminator, generating constructs pEBA528, pEBA529, pEBA530, pEBA531, pEBA532 and pEBA533, respectively. In addition, the A. nidulans gpdA promoter, represented by SEQ ID NO: 6 was cloned upstream of the FLAG-tagged R. emersonii glucoamylase coding region (AG-FLAG) with A. nidulans amdS terminator in Te pep.bbn generating construct pEBA540. The amino acid sequence of AG-FLAG and the AG-FLAG coding region with A. nidulans amdS terminator are represented by SEQ ID NO: 18 and 19, respectively. A schematic representation of pEBA528 is shown in FIG. 5, which is representative for pEBA529, pEBA530, pEBA531, pEBA532, pEBA533 and pEBA540.

### Example 4

### Inactivation of the ReKu80 Gene in Rasamsonia emersonii to Improve Gene Targeting

Cloning of ReKu80 deletion constructs Genomic DNA of Rasamsonia emersonii strain CBS393.64 was sequenced and analyzed. The Rasamsonia emersonii Ku80 gene (ReKu80) was identified. Sequences of ReKu80, comprising the genomic sequence of the ORF and approximately 2500 bp of the 5′ region and 2500 bp of the 3′ flanking regions, cDNA and protein sequence, are shown in sequence listings 20, 21 and 22, respectively.

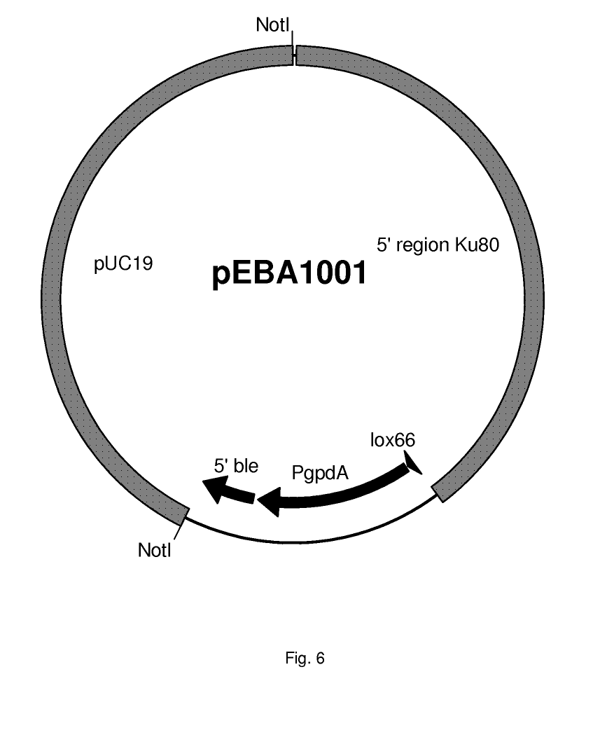

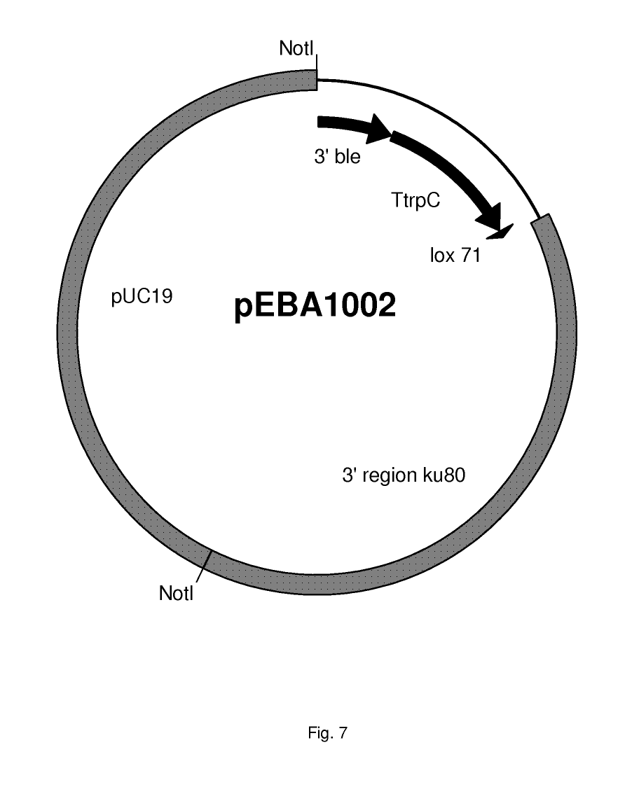

Two replacement vectors for ReKu80, pEBA1001 and pEBA1002, were constructed according to routine cloning procedures (see FIGS. 6 and 7). The insert fragments of both vectors together can be applied in the so-called “bipartite gene-targeting” method as described in Example 3. The pEBA1001 vector comprises a 2500 bp 5′ flanking region of the ReKu80 ORF for targeting in the ReKu80 locus, a lox66 site, and the 5′ part of the ble coding region driven by the A. nidulans gpdA promoter (FIG. 6). The pEBA1002 vector comprises the 3′ part of the ble coding region, the A. nidulans trpC terminator, a lox71 site, and a 2500 bp 3′ flanking region of the ReKu80 ORF for targeting in the ReKu80 locus (FIG. 7).

### Deletion of ReKu80 in Rasamsonia Emersonii

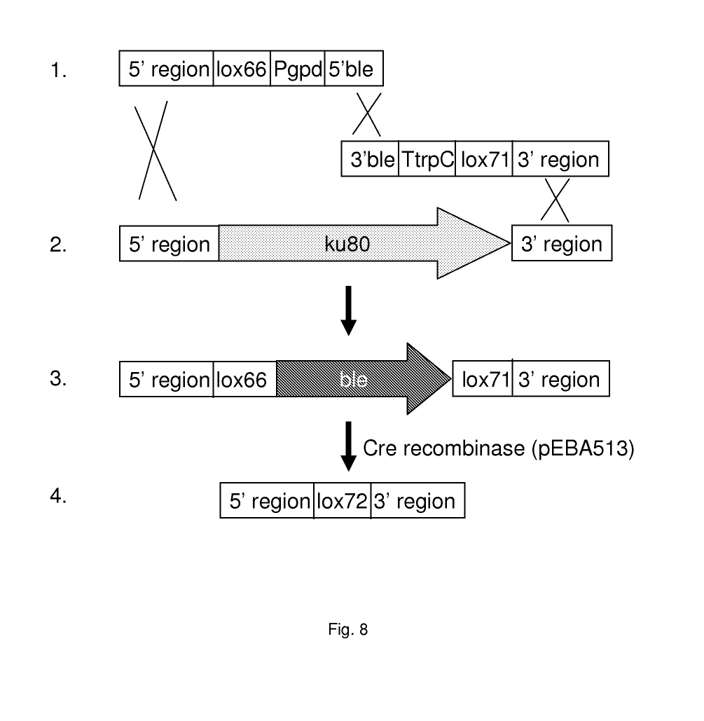

Linear DNA of the deletion constructs pEBA1001 and pEBA1002 were isolated and used to transform Rasamsonia emersonii strain TEC-142S using method as described earlier in WO2011\054899. These linear DNAs can integrate into the genome at the ReKu80 locus, thus substituting the ReKu80 gene by the ble gene as depicted in FIG. 8. Transformants were selected on phleomycin media and colony purified and tested according to procedures as described in WO2011\054899. Growing colonies were diagnosed by PCR for integration at the ReKu80 locus using a primer in the gpdA promoter of the deletion cassette and a primer directed against the genomic sequence directly upstream of the 5′ targeting region. From a pool of approximately 250 transformants, 4 strains showed a removal of the genomic ReKu80 gene.

### Cloning of Transient Expression Plasmid DEBA513 Encoding Cre Recombinase

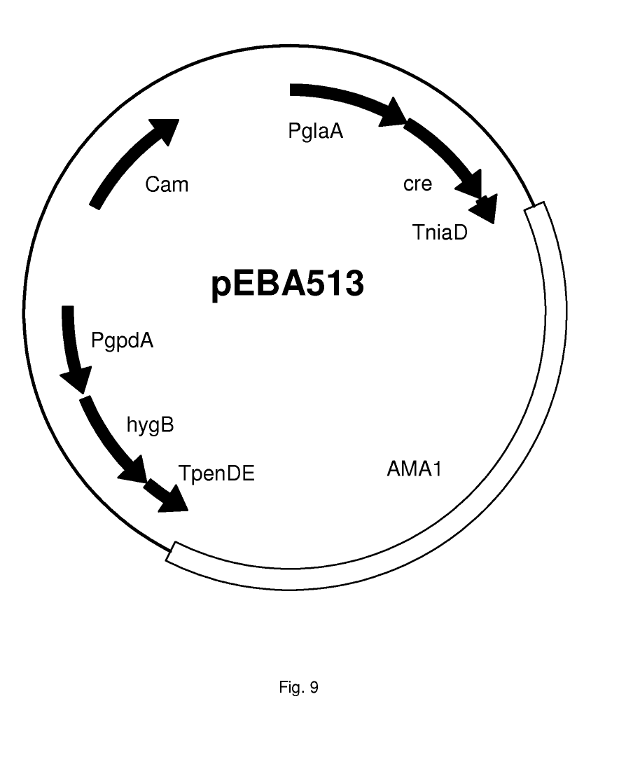

pEBA513 was constructed by DNA2.0 (Menlo Park, USA) and contains the following components: expression cassette consisting of the A. niger glaA promoter, ORF encoding cre-recombinase (AAY56380) and A. nidulans niaD terminator; expression cassette consisting of the A. nidulans gpdA promoter, ORF encoding hygromycin B resistance protein and P. chrysogenum penDE terminator (Genbank: M31454.1, nucleotides 1750-2219); pAMPF21 derived vector containing the AMA1 region and the CAT chloramphenicol resistance gene. FIG. 9 represents a map of pEBA513.

### Marker Removal of Phleomycin Resistant ReKu80 Deletion Strains by Transient Expression of Cre Recombinase

Subsequently, 3 candidate ReKu80 knock out strains were transformed with pEBA513 to remove the ble selection marker by transient expression of the cre recombinase. pEBA513 transformants were plated in overlay on regeneration medium containing 50 μg/ml of hygromycin B. Hygromycin-resistant transformants were grown on PDA containing 50 μg/ml of hygromycin B to allow expression of the cre recombinase. Single colonies were plated on non-selective Rasamsonia agar medium to obtain purified spore batches. Removal of the ble marker was tested phenotypically by growing the transformants on media with and without 10 μg/ml of phleomycin. The majority (>90%) of the transformants after transformation with pEBA513 (with the cre recombinase) were phleomycin sensitive, indicating removal of the pEBA1001 and pEBA1002-based ble marker. Removal of the pEBA513 construct in ble-negative strains was subsequently diagnosed phenotypically by growing the transformants on media with and without 50 μg/ml of hygromycin. Approximately 50% of the transformants lost hygromycin resistance due to spontaneously loss of the pEBA513 plasmid.

Candidate marker-free knock-out strains were tested by Southern analysis and PCR for deletion of the ReKu80 gene. Marker-free ReKu80 deletion strains were obtained and a representative strain was used for targeted integration of promoter-reporter constructs (Example 5)

### Example 5

### Replacement of the RePepA Gene by Promoter-Reporter Cassettes in Rasamsonia emersonii

Linear DNA of the deletion constructs Te pep.bbn and pEBA1006 were isolated and used to transform the ReKu80 deletion strain obtained in Example 4 using method as described earlier in WO2011\054899. These linear DNAs can integrate into the genome at the RePepA locus, thus substituting the RePepA gene by the ble gene as described for ReKu80 gene in Example 4, except that not only the RePepA ORF but also approximately 1500 nt upstream of the start codon was deleted to also remove the RePepA promoter. Transformants were selected on phleomycin media and colony purified and tested according to procedures as described in WO2011\054899. Chromosomal DNA was isolated of transformants to determine correct integration at the RePepA locus by PCR using a primer in the AmdS terminator of the ble cassette and a primer directed against the genomic sequence directly downstream of the 3′ targeting region. Spore batches were generated of transformants that showed deletion of the RePepA locus.

Transformants were grown in shake flasks in Rasamsonia medium 1 at 45° C. at 250 rpm in an incubator shaker for 24-48 hours and this pre-culture was used to inoculate Rasamsonia medium 3 containing glucose as C-source (approximately 10% inoculation). Samples were taken after 24 hours and proteins were precipitated using TCA-aceton and separated on SDS-PAGE. Recombint FLAG-tagged glucoamylase was detected by Western blotting using a FLAG-tag specific antibody.

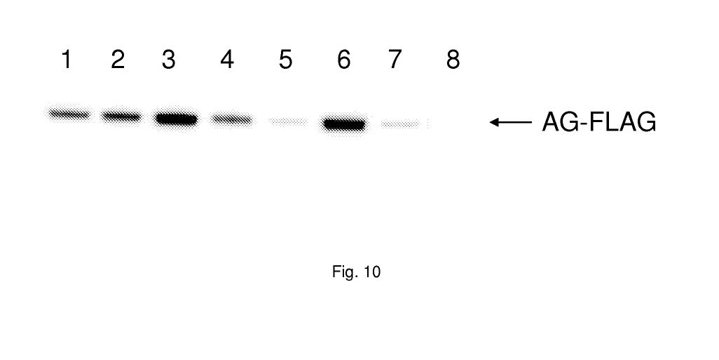

The result of the Western blot is shown in FIG. 10. Supernatants of transformants of all 7 promoter-reporter constructs showed a specific AG-FLAG protein band on Western blot. All of the 6 R. emersonii promoters were able to drive expression of the AG-FLAG reporter gene and some of them showed stronger expression compared to the A. nidulans gpdA promoter in a medium containing 2.4% glucose as C-source under the tested condition (compare lanes 2-7 with lane 1).

1. A Rasamsonia promoter DNA sequence, optionally A Rasamsonia emersonii promoter DNA sequence.

2. A Rasamsonia promoter DNA sequence of claim 1 which is linked to coding sequence which can be overexpressed.

3. A Rasamsonia promoter DNA sequence of claim 1 which corresponds to a strong promoter and/or an inducible promoter.

4. A promoter DNA sequence comprising:
(a) a DNA sequence as presented in the following list: SEQ ID NO:1, SEQ ID NO:2, SEQ ID NO:3, SEQ ID NO:4, SEQ ID NO:5, SEQ ID NO:12, SEQ ID NO:13, SEQ ID NO:14, SEQ ID NO:15, SEQ ID NO:16, or SEQ ID NO:17,
(b) a DNA sequence capable of hybridizing with the complement of the DNA sequence of (a), or
(c) a DNA sequence being at least 50% homologous to a DNA sequence of (a).

5. A DNA construct comprising a promoter DNA sequence according to claim 1 and a coding sequence in operative association with said promoter DNA sequence such that the coding sequence can be expressed under the control of the promoter DNA sequence.

6. A host cell, optionally a fungal host cell, comprising the DNA construct according to claim 3.

7. The host cell according to claim 4, wherein the host cell is a cell from the genus Acremonium, Agaricus, Aspergillus, Aureobasidium, Chrysosporium, Coprinus, Cryptococcus, Filobasidium, Fusarium, Geosmithia, Humicola, Magnaporthe, Mucor, Myceliophthora, Neocallimastix, Neurospora, Paecilomyces, Penicillium, Piromyces, Panerochaete, Pleurotus, Rasamsonia, Schizophyllum, Talaromyces, Thermoascus, Thermomyces, Thielavia, Tolypocladium, or Trichoderma, optionally from the genus Rasamsonia, Aspergillus, Penicillium, Chrysosporium or Trichoderma, optionally Rasamsonia emersonii.

8. A method for expression of a coding sequence in a suitable host cell comprising:
(a) providing a DNA construct according to claim 3,
(b) transforming a suitable host cell with said DNA construct, and
(c) culturing the suitable host cell under culture conditions conducive to expression of the coding sequence.

9. A method for the production of a biological compound in a suitable host cell comprising:
(a) providing a DNA construct as defined in claim 3,
(b) transforming a suitable host cell with said DNA construct, and
(c) culturing the suitable host cell under culture conditions conducive to expression of the coding sequence, and optionally
(d) recovering the biological compound from the culture broth.

10. A method according to claim 7, wherein the biological compound produced is a polypeptide or metabolite.

11. A method according to claim 8, wherein the polypeptide produced is encoded by the coding sequence present in the DNA construct.

12. A method according to claim 10, wherein the coding sequence present in the DNA construct encodes an enzyme optionally involved in production of a metabolite.

13. A DNA sequence encoding a glucoamylase comprising:
(a) a DNA sequence as presented in SEQ ID NO:23,
(b) a DNA sequence capable of hybridizing with the complement of the DNA sequence of (a),
(c) a DNA sequence being at least 50%, optionally at least 60%, optionally at least 70%, optionally at least 80%, optionally at least 90% and optionally at least 95% homologous to a DNA sequence of (a), or
(d) a DNA sequence encoding a glucoamylase and being at least 50%, optionally at least 60%, optionally at least 70%, optionally at least 80%, at least 90% and optionally at least 95% homologous to SEQ ID NO:24.

14. A glucoamylase having DNA sequence being at least 50%, optionally at least 60%, preferably optionally at least 70%, optionally at least 80%, optionally at least 90% and optionally at least 95% homologous to SEQ ID NO:24.


SEQUENCE LISTING (24 sequences)

SEQ ID NO: 1 - DNA, 1494 bp, Rasamsonia emersonii
atgcgcacttcgtgtagagaataccgtcgtcaattggctgtgttacgcagcttacgccagcagctggcttggacgcatcagcattcagcccgcagcgctgctaattttagcgcggaaccggactagcgcgtgacgtgttaaaccctaaatcccgccagtcctaatctggttacagctagggtttagggtctagggctgaatactaggtcttctgagggctaggctgagggttagggctgagtttggtcactgggcatcgctgccattagctttctcttttctccattaggaaaacagtgtggccatccctaatctcaatgacggtgagtttgaaaagttaccgaactgcagtagacgatctttgcacctcaaaaatcagaatgccgaggtgcgcacacgcttggaggtgatgatcgggcatgatgacctcctctgtcagcaatctggcatgaacatcgtcaggacacggtagagagagtggaggacgacgaaaatctgaacagctcgcggttaacatcgcatatccgctagcgccgttgagaacgacacccatcggaatgtcgggaactccaaggtgacagtgctggatgggtcacggctgtcttcaaaggccaagtgccggtcttatgcctatccgcttgagaatgtcttcccgaacaccatcagctttggaaggggctcaccgagagccttcaaccttattagatggtctcgattctgagttacagaattaacgtttcgctgattgttttcacatgacgagccatgtaacgacgcgctgctggaaaagcatagacagacatcgccgtttatccggatcacaggctggtcaattgctctgcgtgtggcactccctctgtaacctcatgatggtctattcgggttatgtaacagcacattatctcagatgaagcatgcaggtaagaaacattattcctctagccaagcatctgcgcgagtggcaagctggaccaggtccccgcatggccattgacggaatttagtcgcaccccgcattgcgggatcgacaatctttactacgcctggcgtgtttggtccctgagacgaaccatcctgccaagctaccccttcttcagcttaaacagtcgtccaatcgtcactggctgttgagcagcatcccccgcatcggcacttgactttaatttcccgatgtcccttatctgagccagacatgcggagtgtcgggagatgagcgatatcctcagactgactgaccagcaagcaagaacgatcgatcactctggcgatttcccgacacggggagtctcgcacttcaggaacaatgcaccggcaactcaacaatggcatcgcttcccaggaatcgatgcccaattcgggatggagatgtcgatcagatggtgtataaaagggcaggatgaatccagggtccaggccatctgccatcactcagactccaacactcgatcagcagcttcgaaagtggtctttttgctatcatccaccgtcaaaatg

SEQ ID NO: 2 - DNA, 1482 bp, Rasamsonia emersonii
atcgtgctgtcggatgtctaccccatcgccgtcaatacaacctggtctgtccaatatcacacggtatgcaacaccacgtacggatgctgtggctgcgacgactgcaagggcgactttgaagacatctcgacgcgtctcgatctgttcacccagtaccaggactggattggaggcccgcccaagaccctctggggcgtcccgcaggcctttgggggtggagagttctgggcgcggaatccaacgccggacgaggaagtgacgatgaacctgctcagcatcaaccacaacgcgaaggggatcgtcatgtggagctacccgacggagccggatctcgaggacgtgaccagctcgctgagcaaggtccttgctgcgccgaacgtgacaagctttcttctaggagcccccaccaccgcgttgaagacggcaggtgcagatcgaatcgacgctgccgggtggcaggtcggaaagcagatgctcgtcagcattttgtatctggagtacatcgattccgattctcaggtcacgatctctttgcccacgccggccagctcagtcacccaagttttgtggggtactggctggactgtgtcagggggcaatctcgtgaagaatggcctgaagggtctagaagcagatttactcattgtgggattggagtaatctagtcatatacatgcttagtttacttggctaatatctgtggctatcaataaatctgttttccggaactttaattctactttatcaattagcctaaaatcatttagacatatgctttttttcccctcatgcaatgcctataacctttccccgcagcaaagccaagcactatcaacaatatccccgattctccgcgataatacataccaaaaatgaaaacggcattttagccaattgacgatctaccctactaaacgttaattggctacctacataagtacctggccacataatttagccagtcgctgtaaactacctaggtatactaggtacgtctacctggagtggagtgatgggctttttttttcaagggggacatctgactaacctttaggtcaacgacctagctaggcagtgaggtacgttaagcggggaaccgggtgtaacgcgaatccggggaagcctccgtggacatggatatagcttgtacagtacagctgtggagatatcccgcatatttttttaaagattaattacaacagccgtacatatacgtcttcatccgctcgtccggaagagctagctaggttacatttctgcttttccagtggggactgtcaaatatgtctttggtaggggaaaaaaaaaacaaagctctttctctcccgagttccctacatataaggatgcagtgcaaccggcgatctcttccattctaaatcacggcaattctctgcacttcccaggtaggaagcatctctgacaattatttgcccttccttctaagagaaagggagagagagaacaaaaaaattcaaaatg

SEQ ID NO: 3 - DNA, 1503 bp, Rasamsonia emersonii
actactgcaagggcgagacggttgtggctgttgcgacgatgcagagtgacccgatcatggtcaagtgtgcggagctgatgcggcgggggaacatgccgtcgaagaaggagattgaggatggggtggatgtgctggctgtgggcgtgcccaagagcgttgctatataagaagccagcgatgtatatacgcagatattttgggtacatgttagacatgttatactctataccatctgtctatgaattcgatatcttgcctcagaatataaccctactatctgtattatatagtgtctaaggcattcaatatgaactcttcttcgatataatgcattaattgctagctgttaggaaacaaactgtactgctagatagatgcttagcaactatctcagagaaccgcacctgggcaaaggtattgttaacgcagtcaattattggcagtgtttgagggcttgcgatctggctgtgaaagcctgcagctacctcagacgatgaagacgatggaaatccatcaaacataggaaggatgtcttcaaaggcttgttatattataaaaggactatattctgttcttcgtttcttaggtagttacctacgtacagtacgtaattcttcccattaataggttcgttagcatgccaaatgactgcacatccccagccattggcaggaggaatctctcccataattaattaattacatctaatctaagtctaaaaatcccacctgattatcaaccaaagagattaatctaaacgaatctaaactccgcagcccagcccagaccagaccagaccaagatcgatccgacactccgcgccacggcgctccaccatcacgaaacaatcatcgctttagtccgtttcaagttggtctatttcgaggttaatcagagggatccgcgggatgtcggccccgaaaccctgcataatcttaattaacctcttttttgggttcattcttgtttctcttccttttacctttagggatagatgccctggatcctcggaatgttgtttgtttatattactactgtaatatgtgatattgtattgcattgaattgactcggatgatcggattccagtaaatacggagtaaacgactacgtcctctgagcaatattccacaaatcaattgttgaagttgaagacgtctgtactatccacactgttccatacgctcgatcgacgcggagaaccggctaaattaagatcgattgttgcccagatgatcatgcagccagatgaaacgtccatcaattcatcgcttttgtggcctgatgtcggagtcgtcatgaggtgacgcgttttttcttttcctgatgattatagccagaatgcggggaagctgtcttgtctaacagtctacgagatggaagatgggctatataaacggtctgctttcctttcagccaggttcttttttttctgttctgattcagtcagtcagatagacagacaaagtcaagctgatcccagttcctccattcctgcaacatg

SEQ ID NO: 4 - DNA, 1503 bp, Rasamsonia emersonii
caatcgattacgactgacctgacgtttgtttatagtcctgagaagcttgtgtggcgggactgctttctgcctgaatatcgggggccgcatctcaaggctagattgaggaagtgtctcgtctgaactagatttgctacatctcaggaggagatgctcagtggaatgttcctgaggcaggctctcttgtgttttgtcagtacaaaccaaactcatagagaatcatctaaaatatagcacatgatactgtggccattgcaattggtccttgttatgcagctgagcaatactggcatagcctgcagacaattgtttattgaatgacagcacagcaagagctgcacgaattgaccaatcgttttgtttgttttagtgcactgtttctgaaatgacgaaatgaacctatcctactaaccccgcattcaggggctaggaacaaggacaggtagactgattcaatcaatggcttggtccaggttctaggcatgcttccatttatccggtgacccctcacatacagcacagtagtactgtgcaaggtaccaaggtgtctagcaagtacgactacacaatccctaatacaagctctgtatgtagcgtcatatcctcatatctgacgagatatcaagttgcctattcgctatttcactagcctgtaaatcccactaccaaatactaataattaatacgaaaccttctgctcaccggagtcggacattccccgtggggattatccagaaggatcatcggcaattactgcacttgccagcaagcaagcttaattaagctaggaacgaaagcatgcaggtgtatccgacattccgcagtagtttaatttagttaaatttaagtagcctgcgaccccgcatgttaggggatgctgatgcaacggtgcctgtgcctagttagtggattgtttagctagatacgggaataggggaatcatctaataattagcctatgtcatgtgaggattggttcttatggtcttcacatgccattgatgttcctggcagtgggatgaaatggtgtccttgagatagtcgaagattaggcaattggattgtatgttgtttctcgcgtagatgttctagtcatatagaatagggatcaatatgctcagcgtcagctacctcaggtcgatgtaaagcacgaatatattaggatagttcaactaggaaactctaatgtacccagcagtagcaattgaagaaatccgacattcccattgtcatcgaaatcgggcagaaatgtgcaaagagagacgattcggtaggcgtgatcgaactgcaagcttcaatccatagcttagttccagaccatgtcgtttccacaaagctcgatccaagttagatgaaagctactagcagctccgacactccgcgagacagctccatcatccattataaatatggccaataaccctgcagaacagcttcaatctgcagcctgcttgaccgtcaagaacagacatcataatatcaatttcagagagaattcaaaatg

SEQ ID NO: 5 - DNA, 1979 bp, Rasamsonia emersonii
caggactcgaatttacactataacactgttgtactgtacaacaatgtgctctgatccattggtaaattaactcgatctcgatcttctctccacgtgcaaaccaagctcatgtctgcgttaaacgtctttccagaaggttaccttttgaatccgaaacgaaggtagcgacgtgcggtggcgaccaatccatctcccaatatggttatgatcgtacgagatcgtcaggggaattgtcaactgaatgccaatgatagaaaacaggttttctggtgttctcttagaagacaccctctcaccttcacagcaattcccatccacattcaagcggacatccagcgaatgctggcgataacgttgctaatcgaattgtgtaattgtggcaatccgcatgcggcgaaggcattaattactccgtcgccacgaggagggggaaatacgctaaggacagtcaggtatagctgcctccgtactgcctgcctaaaacggcatccccgttgccgatcccccggggtacggccagaccctgcataggaatgacgctgccggcagttgggccagagcagccttgcctctgcgtctggagtatggagtaacagagtacggcatattacagagtacttggttccagtgctaagaataggcatcagtaataaatatttgactttgtcttgctacaagttgcagtggtaaacacgggaactggtgtgctggaaaaccgggacgccataggaatcaggtcacggggcagacggtgacggaaggctcgggctcgggctgtctctcgggttgacttgccagcatcccggctgcctgcagcctggatcaagggaaaacaatcaggcgcattctccgtgagatcgagacaccccgaggacgttatttgccagcaggcttccgaaacaacaatgcccacaacaaacactcgggcaatcgatcggggttgttattgatagaagctcgtggcaaagcctggaagggcgacctgactgccgactaagccgtctctggaatctacggtagcatttcagcgcaatgcgggggggttggccgacccatggaagagagagaacttggaagaaaagtaagtggctttgagctgctggcaagtcctttgctaccgttacacctgtcagtactgcgcggtgaggcttgccccgctgccattgactcgggagtgatgcatgaattaattattagacagcgagggactgtcattaataattaatattttgccgatggaggattgccacgaatttagcctgcaataattactccgtgctgggcgttgttccgtaccttctgcttttgtcagccctagcgccggcgccttccccaggcaacgcaagcccctgctagtcgcggggaagccggagcccggcacagccaacccaggaggaaggtacggcccagaggtacggtgctgcctgcccaggtttctgcagctgcatggcgtgtgctggatagcaatttcctctgctcgccagaagacagccaaccacacgatgcgtcgtctgcagctcccaggaaacccagtggttaggtgttatccttgactctttgacagactcgcctgctccatcgacaacaggaacttgcccagtagccgccttccgagtctctgcgtctccaccgcgtgtccacggccgatccgcccgtatgatctcagacccgtccaccgtgtgttgtaatcccttgtcgatcgtacgccgaccatggggctcccgttctcgatctttttcgggcagacgccattccggtccaagactcctgctaccggccttactagtggcccagggccactggggaccgaccctcatttcccgtgtcggtgcttggtgccattgttgtcctgcccgtttctttttataaaagagcccgtcgtcctccctcctcccaaggcctgaaagagcatgtcagctttcctctgtgcctgacttcttgtgttacctggttcccctcgccgcctcaccgtcaaaatg

SEQ ID NO: 6 - DNA, 803 bp, Aspergillus niger
ccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagccaccgtcaaaatg

SEQ ID NO: 7 - DNA, 6493 bp, Rasamsonia emersonii
ggagttgcttgcccgggcaggatatcggttggcggcgtggttggacttgattgcgaagcagtagagacaggtattctgctgatagcatggatttatattatcaataagcagaggcctaaagacattcaaccagaagaaacgtccttcatgcaacagcaagctagaacatatacatatgagaacaagcatagtgccaatcaagcagacctatactgtactctattacaacaaactactatcacggtagtcgtacactggtcaatgaaataatgtgagtaaaaatgatcatgattctatgacagaacagctagtacgcttgatttattggggtataaattttactttatttaggtggttaagagccagcagactagatatatagctcagatataatataattaatagtcacagaaaaaaataaataaaaataaaaatagcaagatccatgatatggtatacacaaaaaaataataatcataaatcacacaattccatcctctccaaaaccacctagccagctcctaccaaacgatacatactcagtccaagcaaatcccccgttccgtcctcccgtccgtccgatcagtcccgaataccgaccaaaaaaaaaaagagacaaaatccaaatcacgggttcattcacatccccacaggatacccatggatcagtcgtcctacttactgtggtacagattagaacagaaaattaggtttttacacaactcagggtggttgcattgcattgcattgtgctgtggagttagttaacttagttgtactccatccagttcatacgcagtacattattgggcatttgaccccatcagacaagatatctaaggataagagtagaaattaggtaataatagtcaaaagagaagaagagataccaagggaactagatactaacaaccaatcaggatatgccacagtgtggaacagaatggaagcagacaggatcaacataactggaaataaccttttctttctttcttcgtacagcatcttggcaggaagtaacttgatattgttaattaatgtccatgtccatgtccatgtctttgttatgtcttgtcttgtctttcttgtgtgctacaagtacagtgtaacagattcatatccgctgaaacagacataacattcgacacatggaatacggagataaagaaataaattatactatatacggaatgatatcaataaatatccgtgttgtactccttattaagaaagagtggcgcttggcgcgtctacgtgcatggactggtactacatatttgattacttcgatttttaataacaacaacctagtagacgtatgtatgtcatgtgaaactttcgattgcgtgctttcttgtctacttgtcgacttgttacaatcttgtcgaatattaataataataatccatcgcactgacatcttggcaagtactccgtacatcaggttacatacatactgattctctaaagctagataacgaataggattctcgcacagacagtatgtgtctcttgtctgtcagatgataagcagatgaacaaagaaagtataactgcttactacctacatgccgacatttagtcgattcctttcggagaatttattatggattattaatagcataccccgggattggcagaaggggtaaaaggtccgactagacaaggatatccatacagtacataccgttgatacagatcgaatcacatgcatactgctgatggtgtgatgaatccttgaattagacaatcatccagacctgtctggacagagatcctggcactgaacaatccactcattgctatctatcggtactctgtacctgtttcagctgaagcttgccaatcgcagactgccatctgcaactgatcagcgccaggatgcaggtcatgataccccagcgttgttcccgaggtgtcattgcttaaacgcgttaaccagtgtgctaaacgtgctaaacgtgctaaatgctaaactgctgatgctatgcagctgcatcgccgaatctggagaatgcagatcacctgccgacggcgggctccgggcacgtgcacgggggaccccgtaggacagaaacgtccatcgagagtacggagtacggagtattacaagaccctgtccatcagaccctgtccatcgtcattgccaagatctctcattgtttgctgtttcatgctcggatcaccagtggacagcaatgccccgtgaacagcaagccgcatgctggtccgtgtcttgtccgtgtgccgatgtagtattgctaacgagacccagaatggcatcaatgacgttgcggatgacagaatgagggggatcatcagtacgtctgctatcaggatgattatcctacggagtatttactcagctgaagacaggaacaagatcgtctgatggatgaggcccacggccagccagcacagactccgtactcttcagtcttctggatttgaccgttcgacggcgcctccgacgtagcatctcgctagcctgatccttggctgcgcctatcgtcggctcatgcccctgttgatgacggggaagtggagcggcgccgcgataaggttgccttgctaatttagcgcctgcacgctccagccaaaaagaccaatattgaggtcgatcgtctcccctggctccgtgctgctggcctgcgatcgccggcgcgatcataccctgcaatcacgccgccagcctatcacagaccatgcggtccttgcaccatctgggagctcgagctctcctgactgccgtcggggcgtcaatgcgtccggagcctccgacgagggcctctgctcctcgtctgtcctactggagcttgtccgtcagacgtcgcatcctgagccgtgtgctgatatcgccatggctctgacgtgatcgactgcgagcggccggcgaggctataagaagccgcaacttgctgctcgaagtaccgtctcccatccatcgatcagacagtcagcagtcctcactcagtcagtcctcagtcgtccttcaccaccatgggtctgtccaaagccttcgtgtctgcactctcgctgtgctccgccgtcgccgtggccgccccgaccgggccagctcccaacgtgcagttctccctgaagcaggtcgcggtgccccggaccaagcctcgtgcgcccccagctgccgactacgcgcgcgctctggccaagtatggcgctccaattccgtcgtctgtgcggacggccgcgtccggcacgcagagcggctctgcggccaacacgcccgtcgccggcgacagcttgtatctcacgcccgttaccatcggccagagcacgctgaacctggactttgacacgggctctgcggatctgtaagtgtcccaactctcgcaagaacaagaacggagcagctgactcgtccagctgggtcttctccaacgagacgccctccagcgagcgcggcaaccacgccatctacaagcccagctcgacggccaagaagctgaacggctacacctggagcatctcgtacggcgacggcagctcggccggcggcgacgtctaccaggacagcgtctcggtgggcggcgtcaacgcctccaaccaggcggtcgaggccgccaccaaggtcagctccgagttcacgcaggagccgggcgacggcttgctgggcctggccttcagcagcatcaacaccgtcaagcccaagccgcagacgaccttcttcgacacggtcaagtcctcgctcgccaagccgctgttcgccgtcaccctcaagcacaacgagcccggcagctacgactttggctacatcgacagctccaagtacaagggcagcatccagtacacccaggtcgacaactcgcagggcttctggcagttcacggccgacggctactcgattggcggcagcagcggcagcggtggctccatttctggcattgctggtaagaactccccctacatcagagttatctagatgctgatttcgcagacaccggcaccaccctcctcctgctcgacgaccagatcgtcaacgagtactaccagcaggtccagggcgcgcagaacgaccagaacgccggcggctacaccttcccgtgcgacgcgcagctgcccgagctgagcttcaccatcggccagtacaccgccaccgtgccggccgagtacctcaacttccagcccgtgtcgcagggcagccagacctgcttcggcggtctgcagtccaaccagggcattggcttctccatcttcggcgacgtcttcctcaagagccagtacgtcgtctttgactcggacggtcctcagctgggctttgctgctcaggcgtagaccagtcgtcctccagcccaggttggttggtaggagatgatttttcgatcgatcgattatcatggtgattgataggatatgtgcatgagcagttgcctgtacatacatacataatgatttattgaatcaattagttatgatcaatctcgaatatattttcagtgaaatacgtacatggtcatagcataacgatatactccgttttcttcaggtagctagtaaatatacacaaattcatcgttctcccggtccgtcaggtccaggaaggctttgtctccgatcgtcccgtcgggatcactctcgctggtatcgtgatagcgctccctcatcgagtaatcaaccaccttggccggctttccattccgcacgcgccgccgctcctgcatccggttcagcacgaccaaattcgcccactgcgccagcacgacggccaccagcgccacgaagatggccagacaagcccgcacgcccggccgatacgccggcgcgtccttcttgctgaagagcagcgggccgacgatgttgcccgccgagctggccgcgttgtacaggctcatcagcgccgatttcttcgttgtgccgcccgtgttgcccacgatccacgtcacgatcagtgggttgccgccgaagagaaacgcgagcaggtagtagcctaccaggagggagggttcgactgagttttgcttcgtgctgttattactgcgtggcacggcgtacagaattgccaggcccgcgactaccggcagcatgaagccggccagcacgacgcccttcatccgcgcccgctgcgccagatagctccccgccaggatgaccagcagctgcagcgcgccaaacggcatgttgagcagactcgtcgtgtacgcgtcgtaccccaggccgttgaggatcagcgggccgaacgtgttgctcacgctggcgccgacgttcagcagcatcgccatgccgatccagaggtaggttttgggctcgagcgctgcctcgacgacgtgccggatcttgaactcgcggctccctgtgcccgtctggttcgcgcgcagccgctcgatggcctgcgctttttccgtctccgtcaggaaccgcgctgaggggatgtcgttgtctagtttccagtagatgaacggcactgagatgatggtcaggaggccgacggtgaggaagatactgcgccagtcgtcagcagtcagtcagtcagcgttcggggtagaaggggagtacatctgccatggcctcagaacaggcgactcgatatggcccaacccgtacgacagggccgccgcgatgacagtcgccgcgccgttggtactgtaccaggccgcaatgcgcagcggctgctcggcgcggcggtaccactggctggtgatgacgctgaacagcggcagacaggcggcctcgaacaggccgaggaagaagcgcgcggccatcagggaggcgaaactgcgacaggcggccatggcggcctgggcgacgccccagcccagacacagcgcgggcatcaggcggcgatgcggcacgcgcacgatcagccacgacgagaacggctgccagacgagctgggcgatgggcgcgatcgaccccagcagcgagtactggttgcccgtcaggtgcgtgtcggcctgcaagccgaaggtggccccgtacccgagcaccgacttgtccaggatctgcaggaagtacacccacacgaggatggccaggatgacgcggtctgtcttgcgccggatgcgcttgctgtcggcgtccgtgagtgggattctctgctggccgatcaggcggagcgccgtgtcgccgtggacggcgggttgctcttcttcatgggtgacggtcggtttggatgccatggtagcgattactagatgtaatcaagttgtaatgggagacaaacgaccaagttctctctcgacgttttataccggcttatatgtctgttcagcagcattgcaagtcaagtaatgacatcggaattcctccggttccccgcattgcgcggcgatcatcggctggcactagcagtatagctagctcagagtccgtattactggattctattgcattgcgctgattgcagacgttgactgacagcaggagctttgactctattacccccacgcttcggcaattccccgcgtgctcgggcctctatgcacccccacgtgggggaacattccagagtatgcaggcagtagtatgcagcatggat

SEQ ID NO: 8 - DNA, 1194 bp, Rasamsonia emersonii
atgggtctgtccaaagccttcgtgtctgcactctcgctgtgctccgccgtcgccgtggccgccccgaccgggccagctcccaacgtgcagttctccctgaagcaggtcgcggtgccccggaccaagcctcgtgcgcccccagctgccgactacgcgcgcgctctggccaagtatggcgctccaattccgtcgtctgtgcggacggccgcgtccggcacgcagagcggctctgcggccaacacgcccgtcgccggcgacagcttgtatctcacgcccgttaccatcggccagagcacgctgaacctggactttgacacgggctctgcggatctctgggtcttctccaacgagacgccctccagcgagcgcggcaaccacgccatctacaagcccagctcgacggccaagaagctgaacggctacacctggagcatctcgtacggcgacggcagctcggccggcggcgacgtctaccaggacagcgtctcggtgggcggcgtcaacgcctccaaccaggcggtcgaggccgccaccaaggtcagctccgagttcacgcaggagccgggcgacggcttgctgggcctggccttcagcagcatcaacaccgtcaagcccaagccgcagacgaccttcttcgacacggtcaagtcctcgctcgccaagccgctgttcgccgtcaccctcaagcacaacgagcccggcagctacgactttggctacatcgacagctccaagtacaagggcagcatccagtacacccaggtcgacaactcgcagggcttctggcagttcacggccgacggctactcgattggcggcagcagcggcagcggtggctccatttctggcattgctgacaccggcaccaccctcctcctgctcgacgaccagatcgtcaacgagtactaccagcaggtccagggcgcgcagaacgaccagaacgccggcggctacaccttcccgtgcgacgcgcagctgcccgagctgagcttcaccatcggccagtacaccgccaccgtgccggccgagtacctcaacttccagcccgtgtcgcagggcagccagacctgcttcggcggtctgcagtccaaccagggcattggcttctccatcttcggcgacgtcttcctcaagagccagtacgtcgtctttgactcggacggtcctcagctgggctttgctgctcaggcgtag

SEQ ID NO: 9 - protein, 397 aa, Rasamsonia emersonii
MGLSKAFVSALSLCSAVAVAAPTGPAPNVQFSLKQVAVPRTKPRAPPAADYARALAKYGAPIPSSVRTAASGTQSGSAANTPVAGDSLYLTPVTIGQSTLNLDFDTGSADLWVFSNETPSSERGNHAIYKPSSTAKKLNGYTWSISYGDGSSAGGDVYQDSVSVGGVNASNQAVEAATKVSSEFTQEPGDGLLGLAFSSINTVKPKPQTTFFDTVKSSLAKPLFAVTLKHNEPGSYDFGYIDSSKYKGSIQYTQVDNSQGFWQFTADGYSIGGSSGSGGSISGIADTGTTLLLLDDQIVNEYYQQVQGAQNDQNAGGYTFPCDAQLPELSFTIGQYTATVPAEYLNFQPVSQGSQTCFGGLQSNQGIGFSIFGDVFLKSQYVVFDSDGPQLGFAAQA

SEQ ID NO: 10 - DNA, 1066 bp, Aspergillus nidulans
agacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcct

SEQ ID NO: 11 - DNA, 1065 bp, Artificial Sequence
  3′ part of the ble coding region and A.nidulans TrpC terminator
accagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaccgacgccgaccaacaccgccggtccgacggcggcccacgggtcccaggagcttgagatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccggcc

SEQ ID NO: 12 - DNA, 1501 bp, Rasamsonia emersonii
ctgcgcgggagggcagtcacggttaacgcctccatctattccccagagcgagaattcggcccgacgggaccctggagtcgaactcttgtccgaagtcgggatcccgatctatacctgcgaccagtggggtcgagccatgatgatgacgagatgcctcaggttgtaatgagccgccgatagcacatggccctggcatcgacgccacgttgaggaacgaacctgacgagacacgcatacacgcacgttgatacgcatgtgacacaggaagatataccttggtggtctaatctgcctgcccacttcagatcgaggcggggagctgatgtcatcgctgacaggaatgaggtcattcgtaaataaggccttaaaaatgcatctgacaaaaaatcaataatagacctagagatgaatggatgtatttagccatgtcgatccaacgactcttttgatcgagtctacgaactgacaaatcatggtccatatgtggtagtttagtgagaactgaggttctgtcgcccaaggtgttggtctttttttttatccggctaaattagcgagaactgtagtccctcagtgtccaattctcgacgaattctctcccatcctccggtgtacgaagtagacaggatttaggataggcaagactgatattcgcgattgcgccatctcttcagatgtcgattggtgggttggaggatggtcctagccctcgccttccgcgtgccttttgatattcggccgatactgtcgggtgagaagttgaggctttttctgctgatggatatccaagttatgagatatatccggagggaattgagaggaatgatcaggaactgggttcagagggcaccagaggtgtaacctttttaacagcattcgattactggcatatacaacgccgtcttaaatggtatttagagtactgccatgctcccgaacaggaaaagggcaaacaccctcccgactccactcctgcgcgactcgaccgagtccacgcctctcccccggggctcgcaatgacgccatccgtctattcgtccgagatggcttctgatgtgcagattccccctccagaaccatgggtgtcgtgcacgcccaaaagggaatgcgggacacccaccggcggccggatcgcgagtgacaagaaccgcccacgccgcgaagtcgtcatcgccgtccaatgggagccgttctgccgcgggcggtaggcgattgcgtgatcgccgagcgcaggaggagcgcgattcacgacacaccttcggggttcgttgcccagtgaaggttgggaaccggtggagagaggggaggaggggtctgtcgaggggtatcagtctcgcgagcttcttttatttccaggggcgcgtcctgttctggtccagagttgtttctttctctccaacagcttcgtccgcagtcaattcctctgccatcctcctcctcgaatcagacaaccaccgtccttgtcaaaagtttctataccccctccggcaccgtcaaaatg

SEQ ID NO: 13 - DNA, 1501 bp, Rasamsonia emersonii
ccaattattcaaatcttcaacctgggtcctttgcgactagactcggcactatacccacctgagtcgaatctccgctggacgattttttttcttagaagaaaaaaagcttcagaggctaaggattaggcttccgtacggactccatgccctatcagacagagactccgcaactcatctgatctcttcgatggagggaaaatctgctgtttttcgcaaatttccaacccaccagaaacacccagaactgtcacgactcacacgtcctacggtctttgtgtgagtataactgattatattcacagatgggttactcaatgcaggtacaaactttgatcgcgctctttttgggaccgtctatcaaccggcttcgaaaaatggctcgactagccaatctgacaggaaattgcgatgttgcaaccgtgtatacggagtcctctgtacaacctctgccaacctctgccacctcggtacatgtacggagtagctccccgcagccgcgattggatgcattaaagtgggtcaaccgcagtggcttgcagtccgctgcacgagtccgtatgcaataattcttgacacacacgagtgcacataataataggaaagcagacaaactttgagctgaaggctgtcgagcttggcaaattgcaggatctggctagtttcgaagtcgacttcgcgcgcgcagcagtattgcattattgagtgtgacctgctgcgtgggattagcgtcgcaccggccgaaagctagtctcatccaaggctgagcctgagcgctaattaccccggatcagccaagccctaatggatctaatgaggtgcctcctccagcattcggcctgcatggtgcggcgacccctctctccacgtccaataattgctgttgcgcctgtcgaaccctgccaccgcatctttgccgttttactccgagatctgaaaagcctgctgtggatggcagttcgcaatatgcactctcaatcaggtctgtagcatcttttaactattattctattactaattgcttctggaaggcttgtggggtgtggtttgtcatcaagttggctccctgagcgccgcgttgcaatctccacgcgcggttgtacggagtatattcatgcggatccccggggcagagccgtagtgcatgtgacactaatcgatcatccgctcaattggatcctggatttcgaccctggcttgaacatatccaatgatcttccagggacgaaccgacccggtcatgctttgttacctacgtacggagtagcggcctgggtgatggttccggaaggtctgctaaaaggagatcgagtataccccccggggtccgtctgagacttataaagggctctctgcaactctccggccgacttttttcttcattcgacagccatcactcgtttatctggtcgattctgcagacttgcccaaggagcaaagagcatcttcatacgcgcatcatccatctccagctttctctctccaaacatacaccgtcaaaatg

SEQ ID NO: 14 - DNA, 1501 bp, Rasamsonia emersonii
cagtaagtactccccgtacggggtacaatttagcaatttacattacgtgatggaattgtggatgcagtcagggattggatgtagtagtatattaagcgttaaactaactgtattgtttaattaggcactaaggtatgtacctaggcttaatgctgtaaggtagggttttagaagaaagaatgaaacggtgcgtatagagtatggacacggatttgtgcagatatgttatgtcaaagatcatgcaaagccaagggatggacaggctggtatgtattgaccgaatcagcatgctgtccttcacagcagtcagtctaccgagggtattgacttggcagtagccctaaattctattattgtgccatttgcacaaactttaagtgtgtctgccattctgaagaggaataatacaagttaaaacgagaatagaataacaaggctattattatcaggagaaaggttctagatcaaacaaaatccttgcaagcaattaatttacctaggtattttattttatttcttccaactagatagtttatgcatgtgatcgatccggtcattattatgcattcagatacatgctttccacctgactagcagaaccctactccgtacgtagacccctcagaaacatggcgtgtggcgttgcattatttgatggcgcaacgctaatcgcttccaccccatggggttggccatctgtcttgcatggagttgcctgataggtggtgacatgcgctagctgtttagtactcccaacagagtgtagaccatggttaatgatactatactggctggatgacgagaattatgcgattctgacctctagccttgagctgccggaacaactaaccaaaggaaattattgatgagcagacgagatatcatcacacgagattcagggaaatttcaggtaaataccagcctggcagcagcacagtgggacaacgtgtctggtcagcgaatcttgcgatataacctcgatggatggcgtgcagaccctcagccttggcggcagctcgttactgggaaggtttgatccaagtgagacattgctatgctggatcgagacaagtcgctaaccatcatccaggatgcccttcatgcgactgactcagcacgattcgactggcgagcacatgccagccctcttctcagccgcggttgtccgaactgccgaaatgaagctgcgctgtttgtctgccaccgattgggttgatttttgggctgactttctggggatgacccctgatccatgtcgatatcgcgatgtctgcgccggatggcgaatcagggcgacacctagggggggagtgcagtctttgtcgctggctttgtggtatataaagggtgtgaaacccgctgtccacgatcgatcgatcgtccagttcgatcgtccagtctgagaatatcccacaggccgttctctgaactatcttgatcacgctcgttccgcacgctcgctcagtacacaatcagtaccagactctccaccgtcaaaatg

SEQ ID NO: 15 - DNA, 651 bp, Rasamsonia emersonii
catctgccatgaaaatggcttctgggagatattagtgcgatgtatataattcaaatcttgagttacttgcgagcatgtactgtacatacctaggcaccaggatccttcattgatcaatcacgtattatcttatcgataacgagcctgcaaatctcggttcgctcgactgatttacggagtcaagagaaaatactctgatctaggccaagaagtctttattatatactcccaaggcaggcttcacaccatcttcctcgccatacttgacgtatttctacggagtatattgtagaaaagcacgtgacagtgtgcaaaaatggaaattaatgttctgagagtagtactccgtagtgatgattggccatgttcatctaaatcacgtgagagaataccgatgtccgtctagcctcttgcgggtatagtagatagggcacgagacttaaaccctaaaccctaagccctactctcgcgtaccgcactccactcccgcagacggccaaccaaccaaacgctctgccgcgaaaccctccaatttggcttgcacatcgatcgaacttgaccgatccgaccgacaaacgacgaacgacgacgacaacttcgctcccagcaaccacagtcgccaacacccgcagaccacccaccgtcaaaatg

SEQ ID NO: 16 - DNA, 1501 bp, Rasamsonia emersonii
ctgatattatgccaaggctcttattgagcaacttctttaacctacgttaagttgcagttaagcgatagtcctagcctcctaaggtaatatacggagatactccggagtccgattgctttgcagaacactatgaagaggatgcatgcaaacccatgtcttaagggcgcaatccgtgccattatcccataggggttttactttgtctcccttgggggaaacgtgtccaaaataccgtgttttctatatccgtcattgttgattgagaagcctactactgactctttgatcagtcgaaattggatacatgtatttcccagttctgccatgttttacaaaagtggcaggatcctacaaccgatttaccttactgatcgaagcgaaagcactaattcgaacaagtttctcacccctcatccaggacgatataactctaattcctcccccaaattcgtccctcgcaccgctgcaggaactctgcagataactggatagcctcccttccacgtgggggctaagttccgactactccgtagctacatcttgctagacatggatcattccgaacccacctatagtatttcgagaacggtaccttaaactggaactaatcacgtcttggcagaggaattaaagttggggctcgttaagatctggctctcttaatcgagataaggtaacatcggcgatgatccaagatttgataccaaactatggagtacatacgctcagcctcatccgatcgaacccaaattttgtagtttcaattagggtagctgatattttggtaggatcattctagaattacaaaataggccagtatattgctatttcagcagtgctggactatttttaagtcctacccaaattaaagtccacgtgcctttgggacccgcttgcttcgggaaaacctcagaagtacagacgactctgctgattccttaggcaattacttgtactccataatagcctttctatttttgaggtctttgaggcgtttttgattcgaataatgtgatattttcccgtgccaagctcgccaacagtccctacggactgctttgataaagtcggcaccgcggcatgatgccctacatccgcaatgcaggaaccaaattggagactttccaatgaaaagtctcgaaattagcttcagggactagttagttcttccagataggattcgcagaccctgatctgacttgactatagttgccctaaaccagccacattgctaccacgaggtcaaagacatcaaattagtacggaacatcgctctatacgacatcacactgtaaccttttttggttcaacgatggcatcggttagggaaaacatgaggctttctagacatgttctcgatttttttttctgggtatataattgccatggatacatatgtcccatggagtccagaatttcgccagagcagttgcaactttttgctcattctatagttctttctttctcgtgttattattgaattcattgcaccgtcaaaatg

SEQ ID NO: 17 - DNA, 1501 bp, Rasamsonia emersonii
cggtcatttattttctatgatggccatagctaaagtacaggtagtcttgactaagtttacactgccttcgccagaatattcttaacactggcacgtagaggagactcgctgttcacgacgtcatcatttgcaatgaagagttcctcacatgcgcatatattgtttaccagttgcagcgactccgcacctgaatttccaggcattctcaccggagatctaacaatggcaattgttagaagcatccatggatatgcgccgtcccgcaacgttgcagccttggtgccggagcagacggagaaggaaagtaaaatatattagggagcagagtcgcatgtgaggggaacgtgatcttatgtatcatttttcggcgaacaaacgtgatgggccaacatgatcgttcgagttagctcgactggacaagctttagcccaggaaataaggtacggagtaaatgcgaatccgacgccatatcggcaactagcaccgacactatgcaaccctggacgacgccatcatacctccctttactcttcacggcgtactttggacaggggacgggcttttatcatttctccaactccaataattggcagactaagtcttgcgactcttgggggatactcgtgctggatctgaagccggatccagccgggtcgagctgtgcgacacgcgacgcacaggcatcaccaggcaggtcacttgatgctggtcatacaggacaagagaggcacattaccaggtcatgctcgcaacatgagcgtgagcacggactgcgagagattgtcccagccctggaggtgagcttggttgccaatcaactgcggggaggactcatgacaatcgacggtccacggatgatggccatgcagaccagggtgcagcatcaaatgcgattctgctgtgacgccggacggaaatgagatgaccctggcatgcgactccgggcaccccccttgcagggcctcgctctcagccccgcatcccgacttgctgctatccatggatcgcgacaggcatccgcgcggccggactggccaacttcccaattctgcccctgccgcgcgcatcgggtcgctagccccaggcttgtccgcgcatctccatgaactagcccgaacgcctgacgagctactgggacgaggtgacattgagcctctaccatggtcgtgatgataccctccatttacgcgcctgacgtggcccggcaatcgtcatcgcaatgcctcatcctcggggcatttcccaggccaacgtcgcagtcctctgtcgctgtttcctgcagtcgattggcacgcctttgggaattgccccgctgctgccatttctcgggcttccctttccgtggtcgactggctgatggtagacgatagaaggtaatattccacgatgggcacggtgggagattcaaaaacgtgcacatccccgagcgtcactcgggaaagagcctctctccagactctcccgttcgccctgtctttccttaacgaccaccgtcaaaatg

SEQ ID NO: 18 - protein, 625 aa, Rasamsonia emersonii
MASLVAGALCVLGLTPAAFARAPVAARASGSLDSFLATETPIALQGVLNNIGPNGADVAGASAGIVVASPSRSDPDYFYSWTRDAALTAKYLVDAFIAGNKDLEQTIQEYISAQAQVQTISNPSGDLSTGGLGEPKFNVNETAFTGPWGRPQRDGPALRATALIAYANYLIDNGQASTADEIIWPIVQNDLSYVTQYWNSSTFDLWEEVEGSSFFTTAVQHRALVEGNALATRLNHTCPNCVSQAPQVLCFLQSYWTGSYVLANFGGSGRSGKDVNSILGSIHTFDPAGGCDDSTFQPCSARALANHKVVTDSFRSVYAVNSGIAEGSAVAVGRYPEDVYQGGNPWYLATAAAAEQLYDAIYQWNKIGSISITDVSLAFFQDIYPSAAVGTYNSGSSTFNDIISAVQTYADGYLSIIEKYTPSDGSLTEQFSRSDGTPLSASGLTWSYASLLTAAARRQSIVPASWGESSASSVPAVCSATSATGPYSTATNTAWPSSGSGPSTTTSVPCTTPTSVAVTFDEIVSTTYGETIYLAGSIPELGNWSPSSAIPLRADAYTSSNPLWYVTLNLPAGTSFEYKFFKKETDGTIVWEDDPNRSYTVPAYCGQTTAILDDSWQDYKDDDDK

SEQ ID NO: 19 - DNA, 2208 bp, Aspergillus nidulans
atggcctctctggtcgctggtgctctctgcgtccttggattgactcccgccgccttcgctcgcgcccccgttgctgctcgtgcctccggcagccttgactcgttcctggctactgaaacccccattgctctgcagggtgttctcaacaacattggtcccaacggtgccgatgtcgctggtgccagcgctggtattgttgttgcctccccctcccgctccgaccctgactacttctactcctggactcgtgacgcggcgttgactgccaagtacctggtggatgccttcattgctggtaacaaggatctcgagcagaccatccaggagtacatctctgcccaggcgcaggtccagaccatctccaacccctccggtgacctcagcactggcggcctgggtgagcccaagttcaacgtcaacgaaaccgctttcaccggaccttggggacgcccccagcgtgatggacctgctctgcgcgccaccgcgctgattgcctacgccaactacctcatcgacaacggccaggcctccactgccgacgagatcatctggcccattgtgcagaacgacctctcctacgtcacccagtactggaactcttcgaccttcgatctctgggaagaagtcgagggttcgtcgttcttcaccactgccgtccagcaccgtgctctggtggagggtaacgcgctggccacccgcctgaaccacacctgccccaactgtgtctcccaggctccccaggtcctttgcttcctccagtcctactggactggcagctacgtccttgccaacttcggtggctctggccgttccggcaaggatgtcaactccatcctcggttccatccacaccttcgaccctgctggtggatgcgatgacagcaccttccagccttgctcggcccgtgccttggccaaccacaaggtcgtcaccgactctttccgctcggtttacgccgtcaactccggcattgctgagggttctgccgtcgctgttggacgttaccccgaggatgtctaccagggtggaaacccctggtacttggcgactgctgctgctgctgagcagctctacgacgccatctaccagtggaacaagatcggcagcatctccatcaccgatgtctctcttgctttcttccaggacatctacccctccgccgccgttggtacctacaactccggcagcagcaccttcaacgacatcatctctgctgttcagacctacgccgatggctacctctccatcatcgagaagtacaccccctccgatggctccctcaccgagcagttctcccgctccgacggtactcccttgtcggcttctggtctgacctggtcctacgcctccctcctgaccgctgctgctcgccgtcagtccatcgtccccgccagctggggtgagtcctctgccagctccgtgcctgctgtctgctctgccacctcggcgactggtccttacagcactgccaccaacactgcgtggccctcctctggctccggtcccagcaccaccacctccgttccttgcaccacccccacttctgttgccgtcaccttcgacgaaattgtgtctaccacctacggcgagactatctaccttgctggctccatccctgagcttggtaactggagccctagctctgccatccccctccgtgccgatgcctacacctcgtcgaaccccctctggtacgtgaccctcaaccttcctgctggtacctccttcgagtacaagttcttcaagaaggagactgacggcaccatcgtgtgggaggatgaccccaaccgcagctacaccgttcctgcctactgcggccagaccaccgccatcctggacgacagctggcaggactacaaggacgatgacgacaagtaaactaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatattggctgggttgatggctgcttcgagtgcagtctcatgctgccacaggtgactctggatggccccatacca

SEQ ID NO: 20 - DNA, 7890 bp, Rasamsonia emersonii
ggagcctggctcagcatgctcacggactgcaggatgaacacgcgtctccgaaggtgcaaccggggagaagataaccaccgtccttgagggaagaacatctgtcatcatcgaaacagaagcagtctcagtctcggtctcggtctcagtcccgtcatctatagtcgtcacagttgggatcggaaacgtggccgaccagtccctggtcttgtaccaggtgttattctcgtatcgcggcggcgcgatcatgtcgagcgcctcaccaatccttccgaggacatagcccatcctgaacccgccctggaggcgctcgatgaagttgttggcgggatgaacggcggtgaaagggatggcatggaagatgccgaggatggccatggccatggagaacatgaacaggccgaaggggtccatggtgatgatgattcgatcgatatcagacagctgaatatgtattagctttccaaatttcttctatcatgaaattcgacaggaaatagaaaaagaaaggatgctggctttgattaaagagagtagctgggtcttatcacaagagattaaaaagtgtcttaaacaaagacaagacaagacaagcatccgtctgatgcatgagtttcgagatatatataatacaaaggatggattcactgaacagttcgttgacttctgaagaacaaaggctggtctctgcatgcccgagaatagtaacaacaataaagagcaaatacaacgttgtgtacagcaagcgaatgacctgccttgaatgagtctggcagatttattcccccatctactaccttcaagtacctcatcagatggccagcagaaggtgcaagtgggtatatcttctcattcgaagaacttagtgtttagttttctgagcagcaatatagctagttgcaagttagaaaagagtataaagaaccgtttccgcaacaccagctaccctcaaagaaaggaattgaacaaactgggaactaccgattactaccaagctggagggcataggcaatgaacgccaggaattggtaaagactgaagataggaaggtatggtgaatactgcatagtgcaatgtagaccttcagtatcataaggatcatccattcattatagacgctaatataaagtatttctgaaaaaaaatgttggagaggagatcaagtctttttattcaacggctttcaaagactaaaagctggaaaaggcaggctatcatcatgatacctagcatagcataccatgtttgcgtttatcacttcatattcaggacaacctcctgcccaggaccacgccataggcaactgtcgccaggagaccgcccaggttgaccagcgtcgagattccatgcagcttggcaaacttcttgttgagctcggtcatctccttggagtgaggaggcgggtcataactcttcttgccgtcgatcgattctgccagagatcatatatatcagcccgtacctatacagtgtactcgtgcgtactcggcagcagccaaaccggtaggtagtaggcaggtaccaaccttgctgccacctctgcttgataactcccacaaccttcggggtcagatagagcaggttggtcagtcccgaaacgaagacgatcgaaagcggcagcaacaccgtcagacgattctcctcgagaagcacccccgcgaggctagagggcccagttcccaggaccgttctggctcccgggtaggtcagcgctgcgacaacggggagagcgctctgcagggtgaagtagatcgggaacaggctattctggagcgtcgagaactgctgacggggaagcgtcctgaacgcgacggttccgccaacgaaggtctacatatataacggaatgttgtaggagcttttgatgagttatctatcctcctgaccaattgaccagaaacaaaactcaggatgtcaaacctgatagatctcgactcccagaagggtgccgtagctgattgaagtgtgatgtcagccgatctattagtagcagccgtagtactggtgacgcaccttagtatgtggaaggggcccaggatagacatcctgctctgttgtactgatatggaaacacctcgtgactggaacagaactatatgggattatacttagacagataccccactgactgggaattcagagggaagagtaagttgtgttatgctacgggtaggttagagaagctgtcaagcttgggtctcccgagctaacgctagctgcatgtggggcatgttcttatctccacggcccgctcaaacctagatctgcttccaacaaagcacaaatatctatacacacggccttttccgtaaggcccacgcaccttcccgacgtcatgtgcactcgcgtctgccgcgcctcaaaaaggaaatatcacgcgtctgcctggaggcgctccttagtcatagaaagaaacgcatctacgccatgcagtgatttatttatctgacatttccttcctcttcgttgcagcaggagggacagctgacatctcttttgcaaaatggctgacaaggaggccaccgtctacgtgatcgatgtgggaaagtccatggggaggcgccgccatggacggccggtatctgacctggaatgggcaatgcaatatgtctgggacaagattacgacaaccgtatgctgacacttgatccggtctcctggaaattaaattcctgcgttgagaactgacatatcttctgttaggttgccacggggcggaaaacggctacaattggagtggtcgggctgaggacagatggtgagattttaccgtgcccgaatcaggtaaatatgatttactgatgtatctggacagaaacatcgaacgacttgcaggatgatgacagctattcgcacatctctgtctttcaggaaattggacagtatgtgcctcagctgacactgatgactagtgacttttcctcgcatatactaaataaatcactgccagggtcctcatgcctgatctgcgaaaactgcgcgacctgatcaagcctagcaacactgatgaaggagatggtgagttttgcccgtatcttcggactcatttgatttgatattgagacctatctacctatagctatctcctcccttgtcgtcgcgatccagatgatcaccacttataccaaaaagctgaagtatcgacggaaaatcattctcgtgacgaacggggaaggatccatgagtaccgatggtcttgatgagatcgtgaaaaagctcaagtccgatagcattgaattggtggtcttgtatgtttttcacttctctttgacttttcttgtggctggtatgcaaaatggctaaactggtttcgttgcaggggtgttgactttgatgatcctgaatttggtgtcaaagaggaggacaagaatccagcaaaagtattcaatgttttttttttagcaggttggaagagttgctgattcgatctgccgcaggctgagaatgaagcggtcctcagaggtctcgttgattcctgcgacggagtctacgggacattacaacaggccatattggagctggacacaccgcgtgtgaaggttgttcgtggaataccctcctttagaggagagctccgactggggaaccctgaagagtattcgtctgcccttcgtatcccagtcgaaagatactaccgaacttatgttgccaagccgccgacagcgagctcctttgtcctacgatctgacgctgcagctggtcaagagggtgcagagaatgcactgacaagcgtccgaaacgcacggacatatcacgtcagtgatgagtccgcaccaggaggcaagagagacgtggagcgagaagatctcgccaagggctacgagtatgggagaaccgcggtgcacattagtgagtccgatgagaatatcaccaaactccagacgaaccctggtctggaaatcatcggcttcattcagagtgaccatgtatgtttctcgtcaagggtatctcatctgaaccgtgattaacctaggatccagtacgaccgatacatgcacatgtctaccagcaatgtcataattgcacagaaagcaaacgaaaaggcgatccttgctctttcatctttcattcacgccttgttcgagttggactgttatgctgtggccagacttgttaccaaggacaacaagcccccactcatcgtattactggcaccatctattgaagcagactttgaatgtcttctagaagtccagctcccttttgctgaagatgttcggtcgtaccgtttccctcccttggacaaggtggtcactgtctctggaaagacagtcaaagagcaccgacatctcccaagtgacgaattgctgaatgcgatgagcaaatacgtcgacagcatggagctcgtcgacaaggatgaaaacgggtgagtcatcacagggaaaccgtcatgctgctcatctcaagtatactgacaactccacagagaaccagttgacagcctggctcccagactggaggattcgtactctccactgctgcacaggatcgagcaagctatccggtggcgtgccatccatccaaacgagcctcttccgcccccttctgagaagttgacgcagctgtcacgaccgccagcagatctgcaagcgcgcgcgaagaaatacctggatcgggtcattgccgccgccgatgtgaagaaaggtctgtcaacttctacgctcccccagaatgcatctgactaaaaaatgctgcacagttccaccaaaagcaaaaggtcgcaagcggaatcgcgaagccgacaaacccctatcgggtcttgacgttgacgagctccttcgtcgcgagaagcgcgccaagatctcagccaacaacgccatccccgagttcaaacagtcgctggtcaacgccgagaccatcgacgccgtccgtgacgcagtcagccagatggaaagcatcatcgagaaccacatccgaagcagctttggagacgccaactacgaccgcgtgatcgaggagctgggtgtcctccgcgaggagctgatcgcctacgaagagccggatctctacaacgacttcctgcggaggctgaaggacaagatcctcaatgaggagctgggcggagacagacgagagctgtggtggctcgtcaggaggcaacgggtcggtctgatagacaagaaggcgtcggaacgggttgaagttactgaacaggaagccagggaggtaagtaagcagatacattattcctttagttccattaaacgagctgcatgatgagctgacttttgttcactagttcatgacctcgaaataaaatagtccattattgctatgtatgtcaaggcgcctggccgtagtagtcttaacatgctgatgctgtgaatcaaagcgccagatgaacaataatagaaataataccacttggtagctgtctccattctcacagatagacaacgttaaagaaaagaaaaacgtaaaaagagggtatatgtggtctagtaacgccgcaaggaaaaaaaaactcatacgttagtttcgaacgcaaatctcaaaatcgagcacttcgagtaaatactctgtcgtatcgtttcgcctcaggatatcttcccgagccttctctttccgatatcgattttccgttgtaatctagttattattactccagttagtaaatgcacgacgggcagtattgtaaataatgaaatcagcagcgagagtacgaacatgtccacatcctcatcggctttccggagcaactcgttctggatctccagctcattgttaatggcgatccccagctccttctgtcgagcgacgattttcatcaactcctccacgctccggtcctgctcttccatcgtctgcttctgcagctggagcacgccctggttgtcgagttcccgcgtcttgtccgtttccttgcccaggactcgtccagaacggggtttggcgctccccaccagggcgtccttgtcctgcatcgaagcgacagcgttgtcgagcttgctcttcgtcaccatcgcgttgtgcagattctccaatccgtccttctctttcttggcgctcgcgatgagatccttcctccgacggatctctccctcgcctaacctgctgccgccccatccagacgacttgtcgctcaggttcttcagcccttcctcaagagcgccgatcatcgaccctgctttcaccaagctgcttttggcctgtgccgagctctcgtgttgtttctgtggagtcgtggcctgatcacgtctcgtcagatgcagcctcgtctcgtgtaagtgcgccttcatctcccggtagcaatccagccacaggactggatccgtgatcggtccgccgcctggcgcgcccggttccgtgatggacttgtgaagcttcgatgcggcagagctatccgacagggcttgggaggggaggtttagaaaggacctccagacgcttgtttgacgccatcgcgggtcctcgctctcgttgatggcccgcaggtatctttccaggcctttgcgccgctcttcgcgcagagtctcgttcgagttcgtgttggaaaaccaggacttcccgggcagagcgacgggtggttgggcgccaacctggcggactagtgcgtcatggaacgatgcaaattctgaatagcgtttctggacaacgaacgaccgtagaggcagccggatggtgatgttgtatagcgtatacggactgggagcgtccgcgatggtggctgtcgggatggaaatttcgacattcggggccatgattatagttcagacgggaaaaagaacaaaacaaagagcaggcccttgttatcgaccaggaagcataattcccgccgcttctcttgcggtatctctgtcgttgcagagttggttgcagagtagtggagtcggccggcgggtggaaactcccgcaatgacgcaggcgccccatcttcttctgccaccgccgatctgtggcttagcttcttcttgtcaagactcgactccaccatcgcgactccaggcagcacgaatcgcacgattgccgaaaaactacaccgtactaggggaaggcctaattaatctattaccctagctaaaaatggggttgtcaaacttatcatatagccgtgcgacccgcccttggaggtcactagatccaacctgcgcacggcctggttacggttgatgggagctaaaattagaacgaaagatatactggcggtccgtccccgcgtctatccacaatccaaaactcgtatgcagagttatctacaggtcgatccaatcatgagtcctttgtgacatgtcgttgaatacatggtctcaatcgagtctgccgttcttacatgaccatcctcaccaagatcaatgtcccgtgattcgactgtcagccaagatacgtctcacctggccccatctctactgtcgacaacgtctgcctatactgtaggtgatcagaatacgcagtcccggggagtctactcgcgatggggtggttcatacgtcggctcctcgtcgacgttgtctctgggtccgtcggagagcgtcaatatagacgggagacgaaagttgctcttgatctatatccatggcttcatgggtgaagaagcgagcttccacaagttccctgctcatgtccataaccttgtcaccattgctctggccgagtcgcacgttgtgtattcgaaggtatatcctcgatacaaatcccgccgagcaatggacattgcacgtgatgatttcagtcgatggtgcgtttgcagactggcatatctctctttagagatcatcctagaaagaaacgcatgatactaagtgtcgaataggctatcaccgcatgagtcggaagatacagatgtgatcctactcggccacagcctgggtgggatcctagccgcagaggttgcgctgctcccatcagcccctgggagcaaggagatcttcgagcatcgtatcctgggactcatca

SEQ ID NO: 21 - DNA, 2145 bp, Rasamsonia emersonii
atggctgacaaggaggccaccgtctacgtgatcgatgtgggaaagtccatggggaggcgccgccatggacggccggtatctgacctggaatgggcaatgcaatatgtctgggacaagattacgacaaccgttgccacggggcggaaaacggctacaattggagtggtcgggctgaggacagatgaaacatcgaacgacttgcaggatgatgacagctattcgcacatctctgtctttcaggaaattggacaggtcctcatgcctgatctgcgaaaactgcgcgacctgatcaagcctagcaacactgatgaaggagatgctatctcctcccttgtcgtcgcgatccagatgatcaccacttataccaaaaagctgaagtatcgacggaaaatcattctcgtgacgaacggggaaggatccatgagtaccgatggtcttgatgagatcgtgaaaaagctcaagtccgatagcattgaattggtggtcttgggtgttgactttgatgatcctgaatttggtgtcaaagaggaggacaagaatccagcaaaagctgagaatgaagcggtcctcagaggtctcgttgattcctgcgacggagtctacgggacattacaacaggccatattggagctggacacaccgcgtgtgaaggttgttcgtggaataccctcctttagaggagagctccgactggggaaccctgaagagtattcgtctgcccttcgtatcccagtcgaaagatactaccgaacttatgttgccaagccgccgacagcgagctcctttgtcctacgatctgacgctgcagctggtcaagagggtgcagagaatgcactgacaagcgtccgaaacgcacggacatatcacgtcagtgatgagtccgcaccaggaggcaagagagacgtggagcgagaagatctcgccaagggctacgagtatgggagaaccgcggtgcacattagtgagtccgatgagaatatcaccaaactccagacgaaccctggtctggaaatcatcggcttcattcagagtgaccattacgaccgatacatgcacatgtctaccagcaatgtcataattgcacagaaagcaaacgaaaaggcgatccttgctctttcatctttcattcacgccttgttcgagttggactgttatgctgtggccagacttgttaccaaggacaacaagcccccactcatcgtattactggcaccatctattgaagcagactttgaatgtcttctagaagtccagctcccttttgctgaagatgttcggtcgtaccgtttccctcccttggacaaggtggtcactgtctctggaaagacagtcaaagagcaccgacatctcccaagtgacgaattgctgaatgcgatgagcaaatacgtcgacagcatggagctcgtcgacaaggatgaaaacggagaaccagttgacagcctggctcccagactggaggattcgtactctccactgctgcacaggatcgagcaagctatccggtggcgtgccatccatccaaacgagcctcttccgcccccttctgagaagttgacgcagctgtcacgaccgccagcagatctgcaagcgcgcgcgaagaaatacctggatcgggtcattgccgccgccgatgtgaagaaagttccaccaaaagcaaaaggtcgcaagcggaatcgcgaagccgacaaacccctatcgggtcttgacgttgacgagctccttcgtcgcgagaagcgcgccaagatctcagccaacaacgccatccccgagttcaaacagtcgctggtcaacgccgagaccatcgacgccgtccgtgacgcagtcagccagatggaaagcatcatcgagaaccacatccgaagcagctttggagacgccaactacgaccgcgtgatcgaggagctgggtgtcctccgcgaggagctgatcgcctacgaagagccggatctctacaacgacttcctgcggaggctgaaggacaagatcctcaatgaggagctgggcggagacagacgagagctgtggtggctcgtcaggaggcaacgggtcggtctgatagacaagaaggcgtcggaacgggttgaagttactgaacaggaagccagggagtccattattgctatctgtctccattctcacagatag

SEQ ID NO: 22 - protein, 714 aa, Rasamsonia emersonii
MADKEATVYVIDVGKSMGRRRHGRPVSDLEWAMQYVWDKITTTVATGRKTATIGVVGLRTDETSNDLQDDDSYSHISVFQEIGQVLMPDLRKLRDLIKPSNTDEGDAISSLVVAIQMITTYTKKLKYRRKIILVTNGEGSMSTDGLDEIVKKLKSDSIELVVLGVDFDDPEFGVKEEDKNPAKAENEAVLRGLVDSCDGVYGTLQQAILELDTPRVKVVRGIPSFRGELRLGNPEEYSSALRIPVERYYRTYVAKPPTASSFVLRSDAAAGQEGAENALTSVRNARTYHVSDESAPGGKRDVEREDLAKGYEYGRTAVHISESDENITKLQTNPGLEIIGFIQSDHYDRYMHMSTSNVIIAQKANEKAILALSSFIHALFELDCYAVARLVTKDNKPPLIVLLAPSIEADFECLLEVQLPFAEDVRSYRFPPLDKVVTVSGKTVKEHRHLPSDELLNAMSKYVDSMELVDKDENGEPVDSLAPRLEDSYSPLLHRIEQAIRWRAIHPNEPLPPPSEKLTQLSRPPADLQARAKKYLDRVIAAADVKKVPPKAKGRKRNREADKPLSGLDVDELLRREKRAKISANNAIPEFKQSLVNAETIDAVRDAVSQMESIIENHIRSSFGDANYDRVIEELGVLREELIAYEEPDLYNDFLRRLKDKILNEELGGDRRELWWLVRRQRVGLIDKKASERVEVTEQEARESIIAICLHSHR

SEQ ID NO: 23 - DNA, 1854 bp, Rasamsonia emersonii
atggcgtccctcgttgctggcgctctctgtgtcctgggcctgacgcctgctgcatttgcgcgagcgcccgttgcagcccgagccagcggttccctggactcctttctcgcaaccgagacgcccattgccctccaaggcgtgctgaacaacatcgggcccaatggcgcggatgtggcaggggcaagcgccgggatcgtggtcgccagtccgagcaggagcgacccggattatttctactcctggacacgcgacgcagctctcacggccaaatacctcgtcgacgccttcatcgcgggcaacaaggacctggagcagaccatccaggagtacatcagcgcgcaggcgcaagtgcaaaccatctccaatccgtccggggatttatccaccggtggcctaggagagcccaagttcaatgtgaacgagacggcttttaccgggccctggggtcgtccacagagggacggaccagcgttgagagcgacggccctgattgcatatgcgaactatctcatcgacaacggccaggcttcgactgccgatgagatcatctggccgattgtccagaatgatctgtcctacgtcacccagtactggaactcatccacctttgacctctgggaagaagtagaaggatcctcattcttcacaaccgccgtgcagcaccgcgccttggtcgaaggcaatgcactggcaacaaggctgaaccacacgtgccccaactgcgtctctcaggcgccacaggtcctgtgttttctgcagtcatactggaccggatcatatgttctggccaactttggtggcagcggtcgctccggcaaggacgtgaattcgattctgggcagcatccacaccttcgatcccgccggaggctgtgacgactcgaccttccagccgtgttcggcccgtgccttggcaaatcacaaggtggtcacagactcgttccggagcgtctatgcggtcaactcgggcatcgcagagggatctgccgtggcagtcggccgctaccccgaggatgtttaccagggcgggaacccctggtacctggccacagcagcggccgcggagcagctttatgacgccatctaccagtggaataagatcggctcgatcagtatcaccgacgttagtttggcctttttccaggatatctacccttctgccgcggtgggcacctataactctggctcctcgactttcaacgacatcatctcggccgtccagacgtatgctgatggatatctgagcattatcgagaaatatactccctcagacggctctctcaccgaacaattctcccgttcggatggcactccgctctctgcttctggcctgacttggtcgtacgcctccctccttaccgctgctgcccgcagacagtccatcgtccctgcgtcctggggcgaaagctccgcgagcagcgtccctgccgtctgttctgccacctctgccacgggaccatacagcacggctaccaataccgcctggccaagttctggctctggcccctcaaccaccaccagcgttccatgtaccactcccacctctgtggctgtaacctttgacgaaatcgtcagcaccacttacggggagacaatctatctggccggctcgatccccgagctgggcaactggtccccgtccagcgcgatccccctccgcgcggacgcgtacaccagcagcaacccgctttggtacgtgaccctcaacctgcccgctggcaccagcttcgagtacaagttcttcaagaaagagacggacgggaccatcgtctgggaggacgacccgaaccggtcgtacacggtcccagcgtactgtgggcagactactgccattcttgacgatagttggcagtga

SEQ ID NO: 24 - protein, 617 aa, Rasamsonia emersonii
MASLVAGALCVLGLTPAAFARAPVAARASGSLDSFLATETPIALQGVLNNIGPNGADVAGASAGIVVASPSRSDPDYFYSWTRDAALTAKYLVDAFIAGNKDLEQTIQEYISAQAQVQTISNPSGDLSTGGLGEPKFNVNETAFTGPWGRPQRDGPALRATALIAYANYLIDNGQASTADEIIWPIVQNDLSYVTQYWNSSTFDLWEEVEGSSFFTTAVQHRALVEGNALATRLNHTCPNCVSQAPQVLCFLQSYWTGSYVLANFGGSGRSGKDVNSILGSIHTFDPAGGCDDSTFQPCSARALANHKVVTDSFRSVYAVNSGIAEGSAVAVGRYPEDVYQGGNPWYLATAAAAEQLYDAIYQWNKIGSISITDVSLAFFQDIYPSAAVGTYNSGSSTFNDIISAVQTYADGYLSIIEKYTPSDGSLTEQFSRSDGTPLSASGLTWSYASLLTAAARRQSIVPASWGESSASSVPAVCSATSATGPYSTATNTAWPSSGSGPSTTTSVPCTTPTSVAVTFDEIVSTTYGETIYLAGSIPELGNWSPSSAIPLRADAYTSSNPLWYVTLNLPAGTSFEYKFFKKETDGTIVWEDDPNRSYTVPAYCGQTTAILDDSWQ